(19) United States
(12) Patent Application Publication
(10) Pub. No.: US 2012/0238008 A1
(43) Pub. Date: Sep. 20, 2012

(54) Rapid Detection of Pathogens Using Paper Devices

(75) Inventors: Charles S. Henry, Fort Collins, CO (US); Lawrence D. Goodridge, Fort Collins, CO (US); Jana Catherine Jokerst, Fort Collins, CO (US)

(73) Assignee: COLORADO STATE UNIVERSITY RESEARCH FOUNDATION, Fort Collins, CO (US)

(21) Appl. No.: 13/420,764

(22) Filed: Mar. 15, 2012

Related U.S. Application Data
(60) Provisional application No. 61/453,042, filed on Mar. 15, 2011.

(51) Int. Cl. C12M 1/34

(52) U.S. Cl. 435/288.7

(57) ABSTRACT

A kit for the rapid detection of pathogens in food supplies. The kit includes a microspot device and one or more indicator reagents to be applied to a well of the microspot device. The employed indicator reagent produces a detectable change upon contact with a pathogen of interest. The microspot device is fabricated from a porous membrane, such as filter paper. A substantially continuous boundary composed of a low melting temperature solid is deposited within the porous membrane extending from the top of the membrane to the bottom of the membrane and defines the peripheral sides of the well. Additionally, a barrier is applied to the bottom of the membrane, thus defining the bottom of the well. The kit can further include growth media for enriching the pathogenic bacteria and instructions for use of the kit employing the microspot device and the one or more indicator reagents.

### CROSS REFERENCE TO RELATED APPLICATIONS

This application claims priority to currently pending U.S. Provisional Patent Application 61/453,042, entitled, “Rapid Detection of Pathogens Using Paper Devices”, filed Mar. 15, 2011, the contents of which are herein incorporated by reference.

### STATEMENT OF GOVERNMENT INTEREST

This invention was made with Government support under Grant Nos. 2009-01208 and 2009-01984 awarded by the USDA, National Institute of Food and Agriculture. The Government has certain rights in the invention.

### FIELD OF INVENTION

This invention relates to pathogen detection devices and methods. More specifically, this invention relates to paper-based analytical devices for the rapid detection and measurement of live bacteria in food and water.

### BACKGROUND OF THE INVENTION

Bacterial contamination of food is a human health threat of global proportions. While the incidence of foodborne disease across the globe may be difficult to assess, the World Health Organization estimates 1.8 million people die of enteric diseases every year. [Food Safety and Foodborne Illness. World Health Organization]. In the United States alone, the Center for Disease Control estimates 76 million cases of foodborne illness occur annually, resulting in approximately 325,000 hospitalizations and 5,000 deaths. [Scharff, R. L. Health-Related Costs From Foodborne Illness in the United States; Georgetown University: Washington D.C., 2010; Scallan, E., et al., Emerg. Infect. Dis. 2011, 17, 7-15] Many species of bacterial pathogens can be responsible for deadly food-related illness, with Escherichia coli O157:H7, Listeria monocytogenes, and Salmonella Typhimurium being three of the most prevalent. [Batt, C. A. Science 2007, 316, 1579-1580; Nugen, S, and Baeumner, A. Anal. Bioanal. Chem. 2008, 391, 451-454; Van Kessel, J. A. S., et al., J. Food Prot. 2011, 74, 759-768] Existing pathogen detection and identification methods and protocols employed by the food industry have not proven to be adequate in preventing foodborne illness. Currently, samples are typically sent to a centralized laboratory for costly and time-consuming analysis. For an industry dealing with high consumer demand and limited shelf-life products, the current food quality and safety assessment process is a major hindrance. Rapid, easy-to-perform and cheap detection technologies are crucial to the safety and cost-effectiveness of the food industry.

Listeria monocytogenes is a ubiquitous pathogen that continues to emerge as a major cause of food-related illness. Initially, L. monocytogenes was recognized as a veterinary disease, but within the last 30 years, foodborne transmission has been identified as the primary route for human disease. [Mead, P. S., et al., Emerg Infect Dis 1999, 5, 607-25; Murray, E. G. D., et al., The Journal of Pathology and Bacteriology 1926, 29, 407-439] L. monocytogenes causes approximately 2,500 cases of foodborne listeriosis that result in 500 deaths annually. [Mead, P. S., et al., Emerg Infect Dis 1999, 5, 607-25] Those at the highest risk of contracting the disease include pregnant women, their fetuses, newborns, and elderly and immunocompromised persons. [Siegman-Igra, Y., et al., Emerg Infect Dis 2002, 8, 305-10]

Listeriosis is diagnosed when L. monocytogenes is isolated from the blood, cerebrospinal fluid or other typically sterile site, such as the brain stem. [Ramaswamy, V., et al., J Microbiol Immunol Infect 2007, 40, 4-13] The incubation period and duration of illness for L. monocytogenes are not well-defined. For example, onset of illness has been recorded within 48 hours to over 90 days from exposure to contaminated food. [Mead, P. S., et al., Epidemiol Infect 2006, 134, 744-51; Linnan, M. J., et al., N Engl J Med 1988, 319, 823-8; Olsen, S. J., et al., Clin Infect Dis 2005, 40, 962-7; (9) Low, J. C., et al., Vet J 1997, 153, 9-29]

Several food types are more commonly associated with listeriosis, including ready-to-eat (RTE) meats, such as deli meats, hot dogs, pâtés and other meat spreads. [Norton, D. M. and Braden, C. R. In Listeria, Listeriosis and Food Safety; Ryser, E. T., Marth, E. H., Eds.; CRC Press: New York, 2007, p 305-356] Uncooked and ready-to-eat (e.g. smoked) fish and dairy products, including soft and dairy sliced cheeses and unpasteurized milk, are also commonly associated with listeriosis outbreaks. [Gombas, D. E., et al., J Food Prot 2003, 66, 559-69] Raw vegetables have also been linked to outbreaks of listeriosis. [Gombas, D. E., et al., J Food Prot 2003, 66, 559-69; Ho, J. L., et al., Arch Intern Med 1986, 146, 520-4; Schlech, W. F., et al., N Engl J Med 1983, 308, 203-6] In the last 10 years, several outbreaks of listeriosis in the United States and around the world have confirmed that ready-to-eat (RTE) foods are a major vehicle of listeriosis. RTE (deli) meats may become contaminated during slicing at retail, and although large numbers of L. monocytogenes may not be transferred to the meat, the pathogen grows at refrigeration temperatures, [Sheen, S, and Hwang, C. A. Foodborne Pathog Dis 2008, 5, 135-46] meaning that even low contamination may result in expansion of the bacterial concentration during storage.

The continued presence of L. monocytogenes in food has necessitated the ongoing need for newer, more sensitive and robust analytical systems capable of rapid detection of this pathogen in complex samples. Borch et al. suggested that because bacteria such as L. monocytogenes can be endemic in the meat processing environment, and since these bacteria are effectively controlled with proper sanitation, L. monocytogenes would be useful as an indicator of the success of processing equipment cleaning and disinfection protocols. [Borch, E., et al., International Journal of Food Microbiology 1996, 30, 9-25] As such, rapid, integrated methods that allow for detection of this pathogen should be developed.

Current methods of L. monocytogenes detection require either a long detection time (24 to 48 hours for cultural methods), or are technically challenging, expensive, and/or require dedicated laboratory facilities and trained personnel. In addition, these methods do not integrate sampling with testing. The ideal detection method should be capable of rapidly detecting and confirming the presence of L. monocytogenes directly from complex food samples with no false positives or negatives.

The need for faster, simpler and cheaper detection methods for pathogenic bacteria is not unique to food protection, but it may also find utility in other fields of public health, water safety, and quality in both developed and developing nations. In response to the need for such detection techniques, a simple detection system using a paper-based analytical device (PAD) has been developed for measuring the presence of live bacteria in food and water. The paper-based microspot device has potential for use as a first level of screening for foodborne pathogens in food processing facilities and water, and could be used in conjunction with slower but more selective culture or molecular-based methods for final identification and confirmation.

### SUMMARY OF INVENTION

Foodborne pathogens are a major public health threat and financial burden for the food industry, individuals, and society. An estimated seventy-six million cases of food-related illness occur in the United States each year. Three of the most important causative bacterial agents of foodborne diseases are pathogenic strains of Escherichia coli, Salmonella spp., and Listeria monocytogenes. The importance of these agents is due to the severity and frequency of illness, and disproportionally high number of fatalities. Their continued persistence in food has dictated the ongoing need for faster, simpler, and less expensive analytical systems capable of live pathogen detection in complex samples. Culture techniques for detection and identification of foodborne pathogens require 5-7 days to complete. Major improvements to molecular detection techniques have been introduced recently, including polymerase chain reaction (PCR). These methods can be tedious, can require complex, expensive instrumentation, they necessitate highly trained personnel, and the techniques are not easily amenable to routine screening. Here, a paper-based analytical device (PAD) is taught for the detection of pathogenic agents, including E. coli O157:H7, Salmonella Typhimurium, and Listeria monocytogenes, in food and water samples as a screening system.

An exemplary paper-based microspot assay was created using wax printing on filter paper. Detection is achieved by measuring the color change when an enzyme associated with the pathogen of interest reacts with a chromogenic substrate. When combined with enrichment procedures, the method allows for an enrichment time of 12 hours or less. The method is capable of detecting bacteria in concentrations in inoculated ready-to-eat meat as low as 10¹ CFU/cm².

In a first aspect the present invention provides a kit for the detection of pathogens. The kit includes an analytical device, one or more indicator reagents, growth media and instructions for use of the kit. The analytical device has a porous membrane having a first and second side and a substantially continuous boundary deposited within the porous membrane extending from the first side to the second side. The boundary defines the peripheral sides of a well, or repository, and is made of a hydrophobic solid. In particular, the hydrophobic solid is a solid at standard operating conditions, such as room temperature, but may be a low-melting temperature solid. The analytical device has a barrier adjacent to the first side of the membrane. The barrier defines the bottom of the well and the second side of the membrane within the region defined by the substantially continuous boundary defines the top of the well.

The one or more indicator reagents of the kit are impregnated within the substantially continuous boundary of the membrane. The indicator reagent produces a detectable change upon contact with a product of a pathogen of interest. The growth media of the kit is adapted to enrich a sample prior to assaying on the analytical device. Lastly, the instructions for use of the kit employing the paper-based analytical device and the one or more indicator reagents details the use of the kit for the detection of a pathogen of interest.

In an advantageous embodiment the indicator reagent is 5-bromo-4-chloro-myo-inositol phosphate (X—InP), chlorophenyl red β-galactopyranoside (CPRG), 5-Bromo-4-chloro-3-indolyl-B-D-glucuronide (X-gluc) or 5-bromo-6-chloro-inositol caprylate (magenta caprylate).

The membrane can have a plurality of wells and a plurality of indicator reagents impregnated individually within the wells. In other words, each well of the plurality of wells is impregnated with only one of the plurality indicator reagents, thereby allowing a plurality of bacterial species to be detected on a single membrane

In further advantageous embodiments the porous membrane is paper, nitrocellulose, polycarbonate, methylethyl cellulose, polyvinylidene fluoride (PVDF), polystyrene, or glass. The hydrophobic solid can be, for example, wax, photoresist, or solid ink.

A low volume of growth media can be provided in pre-packaged, sterile containers. The low volume of growth media can be provided in the following volumes; about 0.1 mL or less, about 0.5 mL or less, about 1.0 ml or less, about 2.0 mL or less, about 3.0 mL or less, about 5.0 mL or less, about 7.5 mL or less, and about 10 mL or less. The instructions for the kit can direct incubation of the sample in growth media for the following time periods; about 12 hours or less, about 10 hours or less, about 9 hours or less, about 8 hours or less, about 7 hours or less, about 6 hours or less, about 5 hours or less, about 4 hours or less, and about 3 hours or less.

In a second aspect the present invention provides an alternative kit for the detection of pathogens. The kit includes an analytical device, one or more indicator reagents, and instructions for use of the kit. The analytical device has a porous membrane having a first and second side and a substantially continuous boundary deposited within the porous membrane extending from the first side to the second side. The boundary defines the peripheral sides of a well, or repository, and is made of a hydrophobic solid. The analytical device has a barrier adjacent to the first side of the membrane. The barrier defines the bottom of the well and the second side of the membrane within the region defined by the substantially continuous boundary defines the top of the well. In an advantageous embodiment the indicator reagent is impregnated within the substantially continuous boundary of the membrane.

In further advantageous embodiments the indicator reagent is 5-bromo-4-chloro-myo-inositol phosphate (X—InP), chlorophenyl red β-galactopyranoside (CPRG), 5-Bromo-4-chloro-3-indolyl-B-D-glucuronide (X-gluc) or 5-bromo-6-chloro-inositol caprylate (magenta caprylate). Similarly, the indicator reagent can be an indicator that reacts with an enzyme selected from the group consisting of β-galactosidase, esterase, glucoronidase, glucuronidase, and PI-PLC.

The membrane can have a plurality of wells and a plurality of indicator reagents impregnated individually within the wells. In other words, each well of the plurality of wells is impregnated with only one of the plurality indicator reagents, thereby allowing a plurality of bacterial species to be detected on a single membrane

In further advantageous embodiments the porous membrane can be paper, nitrocellulose, polycarbonate, methylethyl cellulose, polyvinylidene fluoride (PVDF), polystyrene, or glass. The hydrophobic solid can be wax, photoresist, or solid ink.

The kit can also include growth media. The growth media is provided to enrich a sample prior to assaying on the analytical device. A low volume of growth media can be provided in the kits in in pre-packaged, sterile containers. The low volume of growth media can be provided in the following volumes; about 0.1 mL or less, about 0.5 mL or less, about 1.0 ml or less, about 2.0 mL or less, about 3.0 mL or less, about 5.0 mL or less, about 7.5 mL or less, and about 10 mL or less. The instructions for the kit can direct incubation of the sample in growth media for the following time periods; about 12 hours or less, about 10 hours or less, about 9 hours or less, about 8 hours or less, about 7 hours or less, about 6 hours or less, about 5 hours or less, about 4 hours or less, and about 3 hours or less.

In a third aspect the present invention provides a kit for the detection of L. monocytogenes. The kit includes an analytical device, an indicator reagent, and instructions for use of the kit. The analytical device has a porous membrane having a first and second side and a substantially continuous boundary deposited within the porous membrane extending from the first side to the second side. The boundary defines the peripheral sides of a well, or repository, and is made of a hydrophobic solid. The analytical device has a barrier adjacent to the first side of the membrane. The barrier defines the bottom of the well and the second side of the membrane within the region defined by the substantially continuous boundary defines the top of the well. In an advantageous embodiment the indicator reagent is impregnated within the substantially continuous boundary of the membrane.

The indicator reagent of the kit is impregnated within the substantially continuous boundary of the membrane. The indicator reagent produces a detectable change upon contact with the enzyme PI-PLC of L. monocytogenes. The growth media of the kit is adapted to enrich a sample prior to assaying on the analytical device. Lastly, the instructions for use of the kit employing the analytical device and the indicator reagent details the use of the kit for the detection of L. monocytogenes.

In an advantageous embodiment the indicator reagent is X—InP. The kit can further include growth media. The growth media is provided to enrich a sample prior to assaying on the analytical device.

In a fourth aspect the present invention provides a method of screening for bacteria in a source. The method includes the steps of collecting a sample from the source, inoculating growth media with the collected sample, incubating the sample in the growth media, contacting an analytical device having one or more indicator reagents with the incubated sample, and assessing the reaction between a product of the incubated sample and the one or more indicator reagents.

A low volume of growth media can be used in the method. For example, the low volume of growth media can be used in the following volumes; about 0.1 mL or less, about 0.5 mL or less, about 1.0 ml or less, about 2.0 mL or less, about 3.0 mL or less, about 5.0 mL or less, about 7.5 mL or less, and about 10 mL or less. The instructions for the kit can direct incubation of the sample in growth media for the following time periods; about 12 hours or less, about 10 hours or less, about 9 hours or less, about 8 hours or less, about 7 hours or less, about 6 hours or less, about 5 hours or less, about 4 hours or less, and about 3 hours or less.

The sample incubation time can be a time of about 12 hours or less, about 10 hours or less, about 8 hours or less, about 6 hours or less, about 5 hours or less, about 4.5 hours or less, about 4 hours or less, about 3.5 hours or less, or about 3 hours or less.

In an advantageous embodiment, the assessing step can be a quantitative measurement of the reaction between the product of the incubated sample and the one or more indicator reagents. In further advantageous embodiments the indicator reagent is 5-bromo-4-chloro-myo-inositol phosphate (X—InP), chlorophenyl red β-galactopyranoside (CPRG), 5-Bromo-4-chloro-3-indolyl-B-D-glucuronide α-gluc), or 5-bromo-6-chloro-inositol caprylate (magenta caprylate).

The device can be a paper-based analytical device or a nitrocullose-based analytical device. Additionally, the source can be a source such as food or water. Lastly, the method can further include the step of lysing the incubated bacteria prior to contacting the analytical device.

### BRIEF DESCRIPTION OF THE DRAWINGS

For a fuller understanding of the invention, reference should be made to the following detailed description, taken in connection with the accompanying drawings, in which:

FIG. 1 is a schematic drawing of the fabrication and testing for paper microfluidic devices created using a photoresist process.

FIG. 2 is an illustration of a microfluidic paper-based analytical device. FIG. 2A presents a schematic of a dendritic paper device for detection optimization of flow parameters. FIG. 2B presents a schematic of a single channel flow through system with a large reservoir for pumping sample across the detection zone.

FIG. 3 is a series of drawings illustrating a microspot device according to aspects of the invention. FIG. 3A is a perspective view of a micropot device with a plurality of wells. FIG. 3B is a cut-away view of a well of a microspot device.

FIG. 4 is a series of schematics showing the enzymatic reactions of PI-PLC, galactosidase, and esterase with (A) 5-bromo-4-chloro-3-indolyl-myo-inositol phosphate (B) chlorophenyl red galactopyranoside (C) magenta caprylate, respectively.

FIG. 5 is a series of images illustrating the protocol for ImageJ analysis. In (A) a digitial image of the paper device is generated using a flat-bed scanner. (B) Using ImageJ, the image is converted to 32-bit grey scale. (C) The image is then inverted. (D) The spot area is selected individually, and the grey intensity is measured. (E) The average grey intensity is plotted as a function of the substrate concentration.

FIG. 6 is a series of graphs illustrating the determination of the limit of detection for each live bacterial assay. Pure cultures were enriched overnight with shaking Serial dilutions were made in buffer from the bacterial samples, and each dilution was tested on the paper device for enzyme activity and average grey intensities were measured. The limit of detection for E. coli O157:H7 (FIG. 6A), S. Typhimurium (FIG. 6B), and L. monocytogenes (FIG. 6C) was estimated to be 10⁶, 10⁴, and 10⁸ CFU/mL, respectively. However, enzyme activity and concentration of cells do not directly correlate since target enzyme may accumulate over the long enrichment period.

FIG. 7 is a series of images and associated graphs illustrating the determination of optimal substrate concentrations for (A) CPRG (FIG. 7A), (B) MC (FIG. 7B), and (C) X—InP (FIG. 7C) using the corresponding enzymes. In each paper device, a constant amount of the appropriate enzyme was used while the concentration of substrate (in mM) was increased. A negative control for each assay, in which no enzyme was present, is shown as the first well. The average grey intensity was measured and plotted versus substrate concentration, where each data point represents the average (±standard deviation) grey intensity of four measurements. The optimal concentration was determined from the maximum grey intensity generated for each assay.

FIG. 8 is a series of images and associated graphs illustrating the determination of the lowest detectable amount of (A) β-galactosidase (FIG. 8A), (B) esterase (FIG. 8B), and (C) PI-PLC (FIG. 8C) enzymes using optimal substrate concentrations. Average grey intensities are plotted vs. the amount of enzyme in each spot (±standard deviation of n=4 measurements), and in each assay data are fitted with a logarithmic regression.

FIG. 9 is a series of images illustrating the optimization of the live E. coli assay on the well devices. (A) Equivalent E. coli O157:H7 samples are lysed using various sonication durations (in s) with subsequent colorimetric detection on the paper device. (B) Enrichment volume study with live E. coli O157:H7. Aliquots of E. coli cells were diluted in 10, 5, and 1 mL TSB growth media and enriched for 5 hr and then tested β-galactosidase activity. The bacteria grown in 1 mL growth media gave a more distinct and intense color change, indicating the enzyme was more concentrated.

FIG. 10 is a series of images and associated graphs illustrating enrichment time studies for pure (A) E. coli O157:H7 (FIG. 10A), (B) L. monocytogenes (FIG. 10B), and (C)S. Typhimurium (FIG. 10C) cultures, showing colorimetric results on the paper devices for each assay as well as measured grey intensities±standard deviation for n=4 spots.

FIG. 11 is an image illustrating a cross-reactivity study testing the selectivity of each enzyme-substrate pair. Each row is spotted with a sample containing a single bacteria species and each column is spotted with a single chromogenic substrate. A color change is observed only when the correct enzyme-substrate pair is present.

FIG. 12 is a series of images and associated graphs illustrating an analysis of RTE meat samples spiked with 10¹ CFU/cm², 10² CFU/cm², and 10³ CFU/cm² (A) E. coli O157:H7 (FIG. 12A), (B) S. Typhimurium (FIG. 12B), and (C) L. monocytogenes (FIG. 12C). Samples tested for enzyme activity after 0, 4, 8, 10, and 12 hr of enrichment.

FIG. 13 is a set of images illustrating a microspot analysis of surface water samples spiked with S. Typhimurium.

FIG. 14 is a graph illustrating an ImageJ analysis of surface water samples spiked with S. Typhimurium.

FIG. 15 is a set of images illustrating a microspot analysis of surface water samples spiked with E. coli.

FIG. 16 is a graph illustrating an ImageJ analysis of surface water samples spiked with E. coli.

FIG. 17 is a set of images illustrating a microspot analysis of surface water samples testing for CPRG and X-gluc.

FIG. 18 is a set of images illustrating a microspot analysis of surface water samples spiked with various concentrations of E. coli O157:H7 and enriched for 8, 12 or 18 hours.

FIG. 19 is a set of images illustrating a microspot analysis of surface water samples spiked with various concentrations of S. enterica and enriched for 8, 12 or 18 hours.

### DETAILED DESCRIPTION OF THE PREFERRED EMBODIMENT

A paper-based microspot assay for the colorimetric determination of pathogenic bacteria in food has been developed. Three enzymatic assays have been developed for detection of E. coli O157:H7, L. monocytogenes, and S. Typhimurium with significantly reduced enrichment times relative to standard culture techniques. Implementation of this assay is demonstrated with the analysis of spiked bologna samples, using validation of the method via plating. The paper device is capable of detecting pathogenic bacteria at a concentration of 10¹ CFU/cm² within 8-12 hours, or less, of enrichment, depending on the target species. While this concentration range is comparable to that of standard methods, the detection limits can be further enhanced using specific inducers to drive enzyme production as well as utilizing selective enrichment media to inhibit the growth of competing microorganisms. In further embodiments the device can employ enhanced selectivity of each enzymatic assay, decreased limits of detection, and integration of all three assays, as well as other similar assays for other bacterial species, for multiplexed analysis in a single sample. Importantly, the device provides for a cost-effective, simple, and portable detection device and associated methods that can be employed in numerous industries, including the food industry, as a first level of screening for the presence of pathogenic bacteria, without the need for complicated instrumentation. Using the teachings of the present invention, the ability of the paper-based biosensor to detect three pathogenic bacteria species in a real food sample within 8 hours of sampling with detection levels at the target of 10¹ CFU/cm², a total analysis time substantially less than currently available techniques rapid screening methods is demonstrated herein.

The current ‘gold standard’ for bacterial detection and enumeration remains the culture method. While continuous improvements in sensitivity and specificity have been slowly introduced over the years, including the incorporation of chromogenic agars, the culture approach still remains time-consuming for routine analysis in the food industry. Culture methods require 5-7 days for pre-enrichment, enrichment, selective plating, identification and confirmation, at which point a contaminated product could have already reached the consumer. [Brooks, B. W., et al., Vet. Microbiol. 2004, 103, 77-84; Deisingh, A. K., et al., Analyst 2002, 127, 567-581]

A specific example includes the identification and differentiation of L. monocytogenes in food using the chromogenic agar, RAPID'L. Mono, a more selective agar base than what is used in standard culture methods. The procedure involves enriching a food sample for 24 hours, followed by 24 hours plate incubation. [Lauer, W. F., et al., J. AOAC Int. 2005, 88, 511-517] Finally, the plate must be read meaning a minimum of 48 hours before a result is obtained. Moreover, the standard culture methods are neither simple nor portable making their use at the processing plant level cumbersome and limited.

Molecular-based detection methods have been recently introduced to foodborne pathogen detection protocols as exemplified by polymerase chain reaction (PCR), which can be used to detect pathogens with high specificity and sensitivity. Commercially available systems capable of detecting multiple pathogens are available as a testament to the power of this technique. Despite the wide use of PCR and related techniques, the method is still limited by the need for costly instrumentation and highly trained personnel. [Heo, J. and Hua, S. Z. Sensors 2009, 9, 4483-4502] Additional DNA purification and isolation are often necessary as well, further increasing the analysis time and expense. Furthermore, most PCR-based methods do not provide accurate microbial viability data as the amplified nucleic acids may originate from dead cells. As a result, biological enrichment is used to determine live versus dead cell counts, and overall analysis time ranges from 18-48 hours depending on the bacterial species and the media used for enrichment.

Molecular-based detection methods may provide slightly faster results. However, the required instrumentation is still complex. Recent PCR-based lab-on-a-chip systems [Beyor, N., et al., Anal. Chem. 2009, 81, 3523-3528] and immunoassay-type biosensors [Park, S., et al., BioChip 2010, 4, 110-116; Sippy, N., et al., Biosens. Bioelectron. 2003, 18, 741-749] have also been developed and are attractive platforms due to their compact size and the ability to use sensitive molecular detection. For example, Park et al. introduced a chemiluminescent immunoassay for selective detection of Salmonella Typhimurium in environmental water samples. [Park, S., et al., BioChip 2010, 4, 110-116] The authors showed detection in the 10³-10⁶ CFU/mL range. However, analysis is performed on a lateral flow strip composed of four different membranes that must be functionalized individually for each step of analyte capture and detection. Sippy et al. developed a lateral flow immunoassay on nitrocellulose membranes for capture of E. coli 055, a model organism, with subsequent amperometric detection. Electrochemical detection relied on the consumption of hydrogen peroxide by bacterial catalase, providing 100 CFU/mL detection limits but also exhibiting low capture efficiencies (71%-25%). [Sippy, N., et al., Biosens. Bioelectron. 2003, 18, 741-749]

In 2009, Beyor et al. developed an integrated device for on-chip PCR and subsequent capillary electrophoretic analysis for pathogen detection. [Beyor, N., et al., Anal. Chem. 2009, 81, 3523-3528] PCR in the microchip format is advantageous as it allows for reduced sample volumes and shorter thermal cycles. While the detection limits for E. coli O157:H7 were as low as 200 CFU/mL, the device incorporates complex features fabricated through multilayered glass-PDMS stacking and requires an external power source for operation. While all of these approaches have merits for sensitivity and selectivity, they still require more complex instrumentation and analysis times that are limited by enrichment. A simple visual test that can provide direction for further testing is still needed.

Generally, there are three categories of tests that are used to detect L. monocytogenes, including traditional or culture-based methods, immunological methods, and molecular based assays. Culture-based methods are based on the inclusion of L. monocytogenes specific fluorogenic and chromogenic substrates within solid media. Conventional culture techniques continue to be the gold standard for the isolation, detection, and identification of foodborne pathogens, including L. monocytogenes. However, a disadvantage of these methods is the fact that they increase detection times by hours to days, causing preliminary test results to be delayed. While molecular methods such as the polymerase chain reaction (PCR) provide alternative detection methods that are relatively rapid, sensitive and specific, they require an investment in equipment, reagents and trained personnel.

The first visual paper-based bioassay was developed in 1957, and used to identify the presence of glucose in urine. A strip of paper was impregnated with glucose oxidase, peroxidase, and 3,3′-dimethylbenzidine, dried, and then dipped in urine. Abnormal glucose levels were indicated on the strip by the development of a blue color. By the 1960s, several similar assays had been commercialized, including a multiplex dipstick assay that had three distinct, chemically-coated areas that developed characteristic colors in response to urinary glucose, albumin, and pH. Ten-test dipsticks are now commercially available that test for various biological analytes and these multiplex dipstick tests are widely accepted by the medical community as convenient, inexpensive, and a rapid means of performing routine urinalysis.

The introduction of capillary-driven lateral flow in 1989 eliminated the need for the incubation and wash steps that were a major disadvantage of dipstick-based sandwich assays. Capillary-driven lateral flow also increased the total number of captured and detected analyte molecules, thereby improving sensitivity. These improvements were achieved by fabricating a test strip of one or more layers of porous material, typically nitrocellulose. When wetted with an analyte-containing liquid at one end of the strip, the porous material provided a motive force for the movement of liquid from wet to dry areas of the strip, with the main motive force being capillary action within the pores.

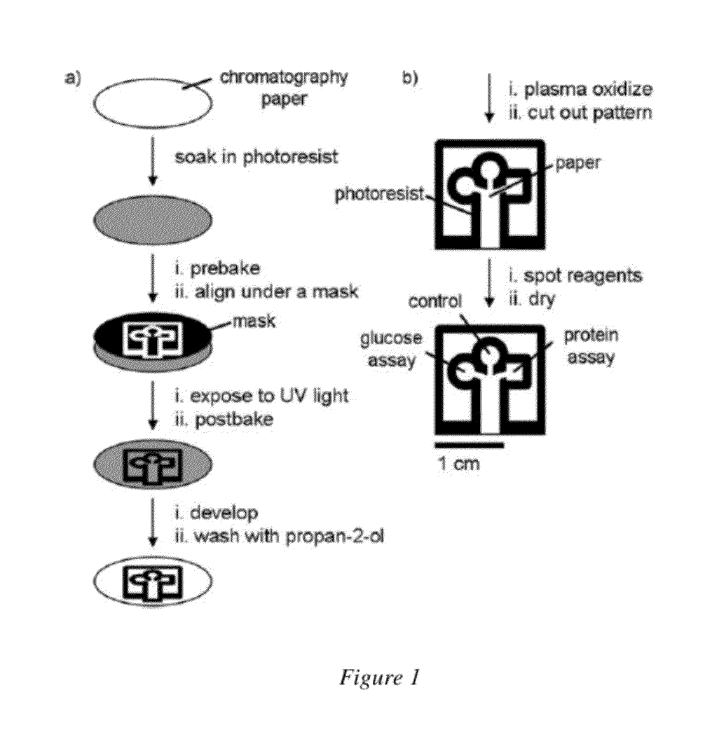

In 2008, a new technology called microfluidic paper-based analytical devices (mPAD) was introduced by Whitesides laboratory at Harvard. mPADs were designed to include the advantages of traditional lateral flow immunoassays with the power of the emerging field of microfluidics [Ohno, K., et al., Electrophoresis 2008, 29, 4443-4453] to create ultra-cheap (<$0.10) multianlayte assays. The basic concept for device fabrication and use is shown in FIG. 1 [Ohno, K., et al., Electrophoresis 2008, 29, 4443-4453] as adapted from the work of Whitesides laboratory. [Martinez, A. W., et al., Angew Chem Int Ed Engl 2007, 46, 1318-20] Here, Whatman #1 filter paper is impregnated with photoresist and exposed to UV light through a simple transparency. The paper is then developed, removing unexposed photoresist. The photoresist defines hydrophobic barriers from hydrophilic flow channels. Colorimetric reagents are dropped on the paper and allowed to dry. Finally, a sample is added at the beginning of the microfluidic channel, migrates to the reaction zones, and reacts with the immobilized reagents to produce a color change. The overall approach has many advantages, including simplicity and the ability to measure more than one analyte from a single drop of sample.

In the past few years, paper-based analytical devices (PADs) have become attractive alternatives to conventional microfluidics as patterned paper is an inexpensive assay platform. [Martinez, A. W., et al., Angew. Chem. Int. Ed. 2007, 46, 1318-1320] In addition to cost, some of the advantages of PADs include small (□L volumes and ng masses) sample and reagent consumption, simple operation and manufacturing, portability, disposability, an extensive application base, a high surface area relative to traditional microfluidics for analyte capture and visualization, and potential for use in scenarios where minimal instrumentation is required. [Martinez, A. W., et al., Anal. Chem. 2009, 82, 3-10] A number of fabrication techniques have also been established for μPADs, including photolithography, [Martinez, A. W., et al., Angew. Chem. Int. Ed. 2007, 46, 1318-1320; Martinez, A. W., et al., Lab Chip 2008, 8, 2146-2150] inkjet printing, [Abe, K., et al., Anal. Chem. 2008, 80, 6928-6934] stamping, [Cheng, C.-M., et al., Lab Chip 2010, 10, 3201-3205] cutting, [Fenton, E. M.; Mascarenas, M. R.; Lopez, G. P.; Sibbett, S. S. ACS Appl. Mater. Interfaces 2008, 1, 124-129] screen-printing, [Nie, Z., et al., Lab Chip 2010, 10, 477-483] and wax printing. [Lu, Y., et al., Electrophoresis 2009, 30, 1497-1500]

The paper-based tool described here consists of a 7 mm-diameter spot array based on a simple well-plate design. Colorimetric assays are conducted in the paper “wells,” utilizing the interaction between species-specific enzymes and chromogenic substrates. Synthetic enzymatic substrates for various microbial assays have been developed that allow for the detection of an expanding range of both new enzymatic activities and target microorganisms. [Orenga, S., et al., J. Microbiol. Methods 2009, 79 (2), 139-155] The presence of pathogenic bacteria is indicated by a color change, a result that may be easily interpreted by the user without the need for complex instrumentation. Additionally, semi-quantitative analysis is performed by measuring the grey intensity of the colored spots using NIH ImageJ software after capturing an image using an office scanner. Such semi-quantitative analysis is provided as an alternative to the more simple visual observation of the color change within the spot. Microbiological techniques employing synthetic substrates for the colorimetric or fluorogenic detection, identification, and enumeration of bacterial species, particularly chromogenic agar media and real-time PCR, have been useful for identification of those species. However, utilizing these chemistries in a paper-based assay has not been realized. [Lazcka, O., et al., Biosens. Bioelectron. 2007, 22, 1205-1217; Manafi, M., et al., Microbiol. Mol. Biol. Rev. 1991, 55, 335-348] In this format, the paper-based microspot test provides simplicity, reduced analysis time, and a cost-effective means of pathogen detection as a tool to indicate the need for further testing. An estimate of the cost of printing a single 8.5×11 in sheet of Whatman #1 filter paper with a wax printer is $0.001/cm². [Fenton, E. M., et al., ACS Appl. Mater. Interfaces 2008, 1, 124-129; Carrilho, E., et al., Anal. Chem. 2009, 81, 7091-7095] Using the 7 mm-diameter spot array, approximately 275 devices can be printed on a single 8.5×11 sheet for an approximate cost of $0.002/device. The total cost estimate of a single spot assay for all three pathogens is $1.35, where the bulk of the expense comes from the colorimetric substrate used for L. monocytogenes determination ($1.28/spot).

Here, use of the microspot test system is developed for detection of three common foodborne pathogens. Such a system can be used to detect bacteria in concentrations as low as 10¹ CFU/cm² when sampled from ready-to-eat meat followed by enrichment procedures, such as those disclosed herein. The overall analysis time ranged from 8-12 hours, or less, including enrichment and detection with the potential to achieve more rapid detection upon improvement of enrichment procedures.

### DEFINITIONS

As used in the specification and appended claims, the term “porous membrane” refers to a sheet or other layer capable of accepting the deposition of wax or other hydrophobic material onto the surface of said membrane and allowing for the diffusion of the deposited wax or hydrophobic material across the membrane responsive to the application of heat or other appropriate force. In an advantageous embodiment the membrane is paper. In a particularly advantageous embodiment the membrane is a filter paper.

As used in the specification and appended claims, the term “pathogen” refers to a bacterium, virus, or other microorganism that can cause disease. In a advantageous embodiment the pathogen is a bacterium. In a particularly advantageous embodiment the pathogen is Escherichia coli, Salmonella enterica, or Listeria monocytogenes.

As used herein, the term “low melting temperature solid” refers to a substance the is generally solid at room temperature and with a melting temperature of approximately 150° C., more advantageously approximately 100° C., even more advantageously approximately 75° C., most advantageously less than approximately 50° C. Wax is an example if a low melting temperature solid.

As used herein, an “indicator reagent” is a substrate that produces a detectable change upon the reaction with a product of a pathogen to be detected.

As used herein, the term “low-volume” with respect to the volume of growth media for growth of bacteria refers to a volume of about 10 mL or less.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” whereever used herein includes the meaning of “and”, “or” and “all or any other combination of the elements connected by said term”.

The term “about” or “approximately” as used herein means within 20%, preferably within 10%, and more preferably within 5% of a given value or range.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mₙ”) or weight average molecular weight (“M_{w}”), and others in the following portion of the specification may be read as if prefaced by the word “about” even though the term “about” may not expressly appear with the value, amount or range. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, the term “composition” is intended to encompass a product comprising the specified ingredients in the specified amounts, as well as any product which results, directly or indirectly, from combination of the specified ingredients in the specified amounts.

Kits for practicing the methods of the invention are further provided. By “kit” is intended any manufacture (e.g., a package or a container) comprising at least one reagent, e.g., a detection reagent such as 5-bromo-4-chloro-myo-inositol phosphate, 5-bromo-6-chloro-inositol caprylate, and/or chlorophenyl red β-galactopyranoside. Such detection reagents may be supplied in a pre-applied form (i.e. “impregnated) on a detection device, such as a microspot device, or may be applied by the user at the time of use and/or testing depending upon the circumstances. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches. In another embodiment, the kit may further comprise a package insert providing printed instructions directing the use of a microspot device and reagents.

Unless defined otherwise, all technical and scientific terms used herein have the same meaning as commonly understood by one of ordinary skill in the art (e.g., in microbiology, cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, ed. E. J. Murray, The Humana Press Inc., Clifton, N.J.).

The following examples are provided for the purpose of illustration and are not intended to limit the scope of the present invention.

### Example 1

### Overview

A paper-based analytical device (PAD) for detection of L. monocytogenes has been fabricated. 5-bromo-4-chloro-myo-inositol phosphate (X—InP) provides a substrate for detection of the enzyme PI-PLC that is released on cell lysis. The combination of X—InP with the PAD allows for the successful integration of enzymatic assays into PADs for detection of enzymes released from pathogenic bacteria, such as PI-PLC. Thus, these two methods can be combined in an interdisciplinary manner to create a low-cost sensor capable of detection of L. monocytogenes. By utilizing additional substrates an array-based sensor can be created that is capable of detecting multiple critical pathogens from contaminated food and water samples in one hour or less.

A reproducible method for device fabrication that is scalable to high-throughput applications has also been developed. Photolithography methods can be used to define the flow channels for PADs as shown in FIG. 1. While this method of production has been successful, it is not ideal for long-term applications and commercialization because it is both time and cost intensive. As an alternative, the use of a wax/solid ink printer, such as the Xerox Phaser 8860 Wax printer, is proposed to fabricate devices. Wax printers are the latest generation of printing technology and use hydrophobic waxes as ink This same ink can serve effectively as a hydrophobic barrier material for printing PADs. Wax-printing is advantageous because devices can be directly printed from a computer CAD program in less than one minute. Over the lifetime of a printing cartridge, the resulting devices would cost ˜$0.04 to print (based on average per page print costs of $0.05, 80 devices per 8.5″×11″ sheet, cost of a sheet of paper, 1,000 sheets printed per year for the cost of the printer of $2,500). The main cost would be in the printer itself, as the ink and paper consumption would be ˜$0.01 per device. Furthermore, because there is no masking process like that used with photolithography, the channel layout can be rapidly changed to improve performance as desired. In other words, parameters such as line width and melting time can be tailored to meet specific needs and produce features of desired sizes on paper.

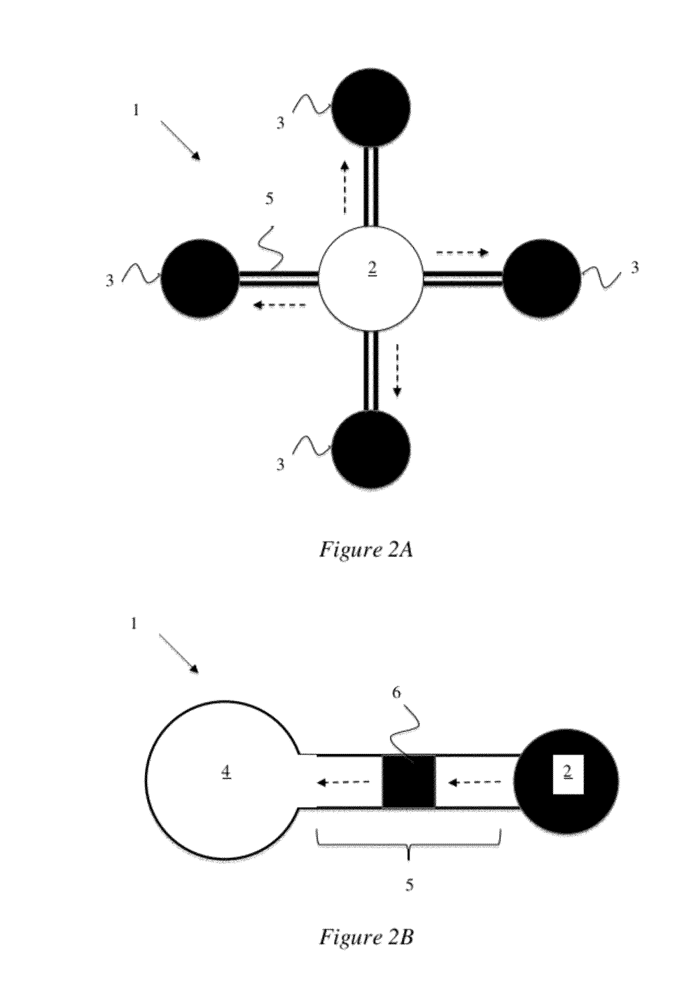

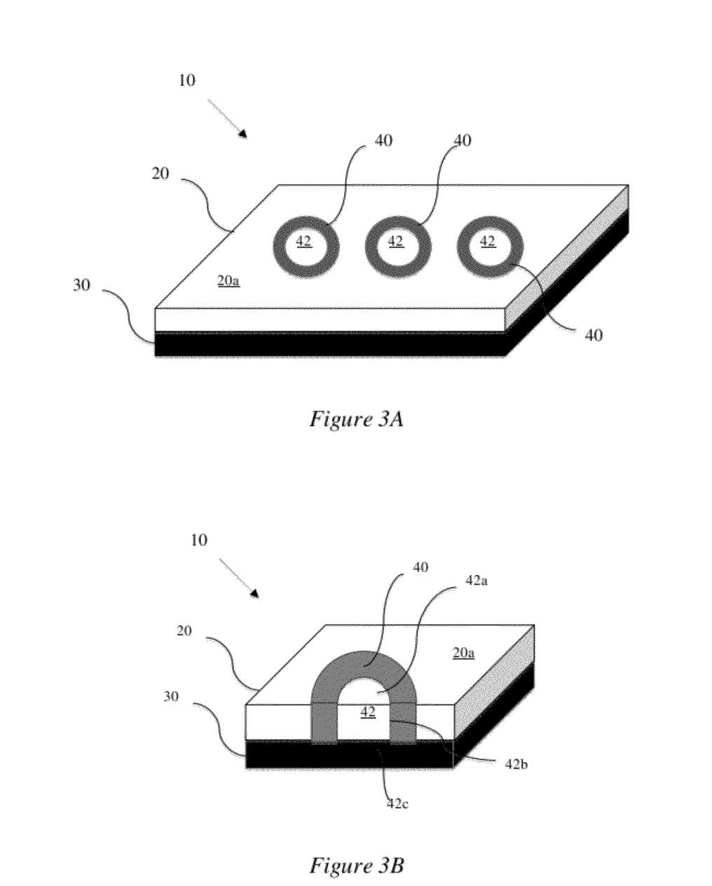

Three different PADS are provided as exemplary. The first PAD is shown in FIG. 3. This design is the most basic and is described more fully in Example 2, below. An alternative design for a PAD is shown in FIG. 2A. The mPAD 1 of FIG. 2A employs a dendritic channel pattern, where the sample to be tested is deposited in the center zone, or well 2, and flows to the outer detection reservoirs 3 along channels 5 via capillary action. The direction of flow from the central well 2 to the outer detection reservoirs 3 is as indicated by the segmented arrows adjacent to the channels 5. The outer detection reservoirs 3 include a dye/indicator reagent that reacts with the sample in the region of the reservoirs 3. Each reservoir 3 can contain a different indicator reagent/substrate, allowing multiple reactions to be performed using a single sample in a single mPAD 1.

A second alternative design for an mPAD is shown in FIG. 2B. The mPAD 1 illustrated in FIG. 2B consists of a single channel 5 and two reservoirs; the first reservoir being the sample reservoir, or well 2, to which sample is added and the second being the pump reservoir 4, to which the sample is pulled via capillary action from the well 2 through the channel 5. The direction of flow of the sample from the well 2 through the channel 5 to the pump 4 is as indicated by the segmented arrows within the channel 5.

As just indicated, sample is added to the smaller reservoir 2 and flows over the substrate, or indicator reagent 6, deposited in the channel 5. The larger reservoir will serve as a capillary pump 4, allowing more sample solution to be pulled over the substrate 6. Commercially available enzyme (A.G. Scientific or similar) dissolved in Listeria-selective enrichment broth is used as the sample for proof of concept. Data is analyzed visually for color development using a desktop scanner with Adobe Photoshop software. Scanner-based reading of colorimetric PADs can result in lower detection limits than simple visualization. In Photoshop, images are converted to grayscale and an intensity map is generated to determine signal. These functions could be also be integrated into a hand-held device if more quantitative detection limits are required. The use media is critical here to determine background specific interferences. With a working system, the analytical performance metrics are determined, including limit of detection, linear range, and background selectivity. Nitrocellulose membranes can be used instead of cellulosic paper where there is concern about loss of enzyme activity on paper. Nitrocellulose is more expensive than traditional paper but is known to maintain protein activity. Another benefit of these PADs, whether constructed of cellulosic paper or nitrocellulose, is that particulate matter from food to be tested should not interfere with the measurements because particulate matter is not transported through these devices.

In testing the devices, as discussed further in the examples to follow, growth media was spiked with L. monocytogenes samples. Lysate from the media was spotted on the PAD and the signal change was analyzed using the methods optimized in the second task. A 4-channel fluidic design (either flow-through or dendritic) provided an advantageous design to test each of the three primary indicator reagents, while leaving one channel as a control. Thus, three of the channels are for positive tests, while the fourth channel is a negative control (i.e. lacking the substrate). The use of a multi-channel system as presented herein also lays the foundation for the detection of multiple pathogens simultaneously. Alternatively, a spot assay could be used with a spot for each indicator reagent and an additional spot for a control.

The ability of the system employing the rapid enrichment of sample followed by detection of the pathogen on the PAD is demonstrated using L. monocytogenes in artificially contaminated food and environmental sources, and detection within 4-8 hours is contemplated. The completed assay is tested on artificially contaminated RTE meats samples. The samples are contaminated with varying concentrations (10° to 10² CFU/g) of L. monocytogenes, and after an incubation period to allow the bacteria to adhere to the meat samples, the samples are homogenized in an appropriate volume of Listeria selective enrichment broth followed by enrichment for up to 8 hours. After enrichment, an aliquot of the enrichment media is removed and placed on the paper assay. For food plant environmental samples, large volumes (5 to 10 liters) of food plant sanitation water are spiked with low concentrations (10⁰-10³ CFU/ml) of L. monocytogenes, followed by concentration for 1 hour using a modified Moore swab. The swab is removed from the device, homogenized in its own liquid, and an aliquot of concentrate is removed and tested using the assay. In both experiments, the paper assays are compared to plating the samples on Oxford Listeria selective medium. The assay is tested for specificity by testing multiple isolates of L. monocytogenes and non-L. monocytogenes bacteria in pure culture. A series of 10-fold dilutions of a cocktail of 5 L. monocytogenes isolates are used to determine the sensitivity of the assay in pure culture.

### Example 2

### Materials and Methods

### Materials.

HEPES, bovine serum albumin, phosphatidylinositol-specific phospholipase C, β-galactosidase, esterase, 5-bromo-4-chloro-myo-inositol phosphate, chlorophenyl red β-galactopyranoside, and 5-bromo-6-chloro-3-indolyl-caprylate were purchased from Sigma (St. Louis, Mo.). Tryptic soy broth, yeast extract, and lambda buffer [100 mM NaCl, 8 mM MgSO4.7H2O, 50 mM Tris-HCl (pH 7.5)] were purchased from Becton, Dickinson and Company (Franklin Lakes, N.J.). Bacterial strains used here were: Escherichia coli O157:H7 SPM0000422 (Lawrence Goodridge Laboratory Strain Collection, obtained from USDA), Salmonella enterica subs. entrica serovar Typhimurium (ATCC 14028), and Listeria monocytogenes FSL CI-115 (1/2a, ILSI, human sporadic). MacConkey-sorbitol agar base, cefixime tellurite (CT) supplement, XLT-4 agar base, Tergitol-4 supplement, PALCAM agar base, and PALCAM supplement were purchased from Remel Inc (Lenexa, Kans.). Whatman #1 filter paper was purchased from Fisher Scientific (Pittsburgh, Pa.). A Xerox Phaser 8860 series wax printer was used for fabrication of PAD devices.

### Device Fabrication.

Paper-based devices were fabricated using wax to define device features and control fluid flow using previously described methods. [Carrilho, E., et al., Anal. Chem. 2009, 81, 7091-7095] Device designs were developed using graphic software, Core1DRAW, and printed using the Xerox Phaser wax printer. Two designs were employed in this work. In the initial characterization and assay optimization studies, an array of 7 mm-diameter circles was printed on Whatman #1 filter paper. Since this configuration of circles conceptually resembles a well-plate, the 7 mm devices were termed well devices. Once printed, devices are placed on a 150° C. hot plate for 5 min in order to melt the wax through the paper, creating a three-dimensional hydrophobic barrier. On the printed side of the paper 2 in-wide, clear packaging tape was placed to enhance control over fluid flow and prevent leaking during the assay, while the reverse side was used for application of reagents and sample. [Martinez, A. W., et al., Lab Chip 2010, 10, 2499-2504]

FIG. 3 presents a pair of illustrations of an exemplary microspot device 10 according to aspects of the invention. As shown in FIG. 3A, the microspot device 10 has a first layer of a porous membrane 20 composed of a material, such as filter paper, that is capable of being printed upon by a solid ink printer (e.g. a Xerox Phaser wax printer). The membrane 20 has a first side 20a, or “top side,” to which sample and reagents are applied and a second side, or “bottom side,” affixed to an impermeable, or semi-permeable, barrier 30. The barrier 30 prevents the diffusion and escape of the sample and reagents as they move across the membrane 20 from the top side to the bottom side of the membrane. One or more circles 40 of wax, solid ink or other hydrophobic material are printed on the surface of the membrane 20 at predetermined locations. The application of heat or pressure to the membrane 20 and/or the printed circles 40 results in the diffusion of the hydrophobic material from the top surface 20a of the membrane 20 across the membrane and to the bottom surface of the membrane thereby forming the sides of a well 42. The wells are completed by the barrier 30, which is affixed to the bottom of the membrane 20, thus forming a bottom of the well 42.

FIG. 3B presents a cut-away view of a well 42 as found on the exemplary microspot device 10 illustrated in FIG. 3A. As can be seen in the figure, the wax from the circle 40 has diffused across from the membrane 20 from the top of the membrane 20a to the barrier 30, thereby forming a well 42 having a top 42a, to which reagents and sample can be applied, sides of the well 42b, defined by the inner circumference of the diffused wax of the circle 40, and a bottom of the well 42c, defined by the barrier 30. The sides 42b and bottom 42c of the well 42 prevent the further diffusion of the sample from the membrane area defined by the well.

### Characterization of Bacteria-Specific Enzymes.

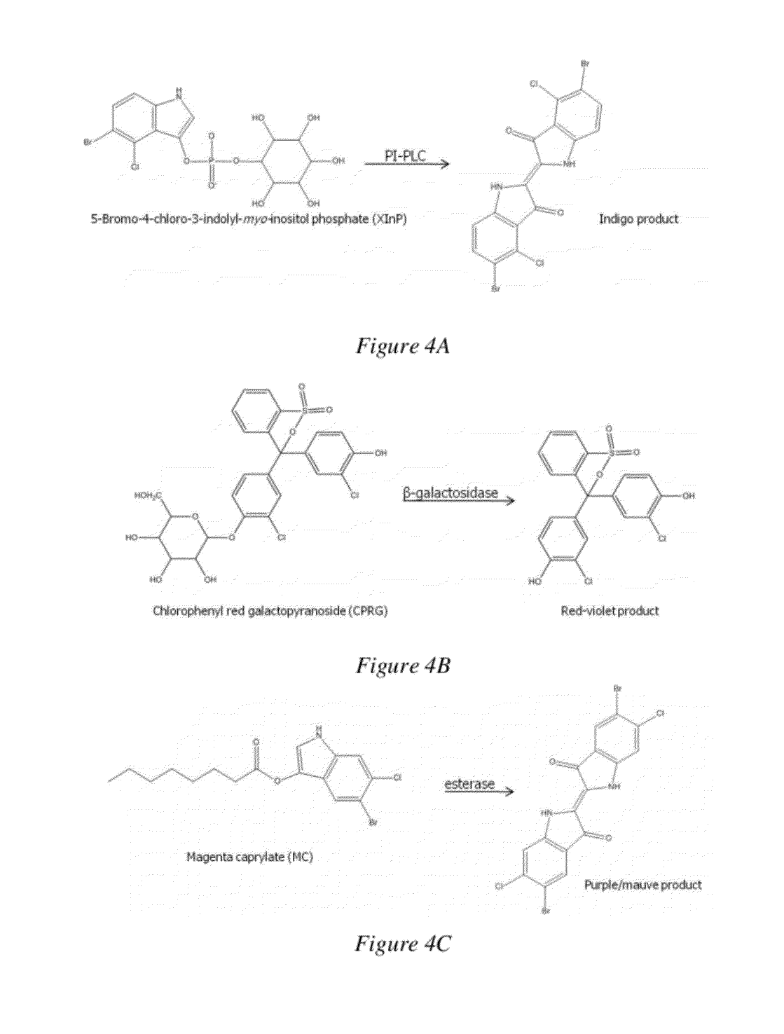

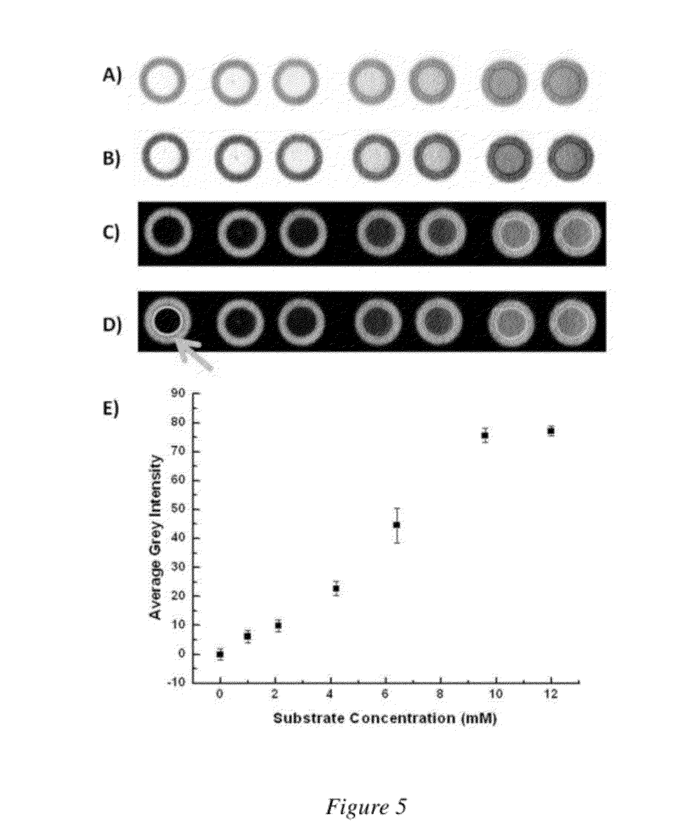

The four enzyme-substrate pairs used in this work were β-galactosidase with chlorophenyl red β-galactopyranoside (CPRG) and B-D-glucuronidase (GUS) with substrate 5-Bromo-4-chloro-3-indolyl-B-D-glucuronide sodium salt (X-gluc) for E. coli determination, [Jacobson, R. H., et al., Nature 1994, 369, 761-766; Tryland, I. and Fiksdal, L. Appl. Environ. Microbiol. 1998, 64, 1018-1023] phosphatidylinositol-specific phospholipase C(PI-PLC) with 5-bromo-4-chloro-myo-inositol phosphate (X—InP) for L. monocytogenes determination, [Notermans, S. H., et al., Appl. Environ. Microbiol. 1991, 57, 2666-2670; Ryan, M., et al., Biophys. Chem. 2002, 101, 347-358; Wei, Z., et al., Proc. Natl. Acad. Sci. 2005, 102, 12927-12931] and esterase with 5-bromo-6-chloro-inositol caprylate (magenta caprylate) for S. enterica determination. [Goullett, P. and Picard, B. J. Gen. Microbiol. 1990, 136, 431-440] In the presence of the specific enzyme, CPRG changes from yellow to red-violet in color. Similarly, X—InP changes from colorless to blue, and magenta caprylate changes from colorless to purple. Initial characterization of each assay involved optimization studies using the pure enzymes that mimicked the enzyme found in the bacterial species of interest. A schematic of each enzymatic reaction, including the final colorimetric product, is shown in FIGS. 4A-4C. Stock solutions containing 1 U/mL concentration of enzyme were produced for the experiments. Aliquots were frozen until use and then warmed to room temperature. Optimization of substrate concentration and buffer pH was determined using an array of 7 mm diameter devices. In all studies, the paper devices were placed in petri dishes upon the application of sample and reagent solutions and kept in a 37° C. incubator. Due to the photosensitivity of magenta caprylate, petri dishes housing this particular assay were covered in foil to prevent exposure to light. Additionally, digital images of the device array were acquired using a Xerox scanner after the spots had dried (approximately 3 hr for drying), and the maximum grey intensity of each well was measured using Image J software. Semi-quantitative analysis using Image J is presented in FIG. 5. While semi-quantitative analysis can be performed for enhanced sensitivity, the device can be applied more simply by visually inspecting the test area for a change in color indicative of the presence of the bacterium of interest.

Live Bacterial Assays.

A number of factors were considered for the detection of β-galactosidase, esterase, and PI-PLC activity from live cultures. For example, in order to free the enzyme from E. coli O157:H7 for subsequent colorimetric reaction with CPRG, equivalent 500 μL bacteria samples, grown overnight in broth, were lysed via probe sonicator. With the sonicator set to 5 W, 22 kHz, various sonication durations were evaluated, ranging from 10 to 120 s. Immediately after sonication, each E. coli O157:H7 sample was tested on the paper device for β-galactosidase activity. Using this method, an optimal sonication time was determined.

In experiments involving pure cultures, a single colony was collected using a 10 μL sterile loop and transferred to a test tube containing growth media, tryptic soy broth with yeast extract (TSB-YE). In an effort to reduce analysis time, various enrichment volumes were studied. A study was conducted to determine the pre-concentration effects of various enrichment volumes for the determination of the species-specific enzymes. An E. coli O157:H7 culture, collected as a single colony, was transferred to 1 mL buffer and vortexed. Next, 100 μL aliquots were diluted in 10, 5, and 1 mL growth media and allowed to enrich for 5 hr with shaking After enrichment, the three E. coli O157:H7 samples were tested on the paper device, and it was observed, a more intense color change resulted from the smaller volume enrichments. Throughout the optimization studies of live bacterial assays, TSB-YE enrichment media was used in volumes as low as 0.5 mL.

For the optimization of the paper-based device with live E. coli O157:H7, L. monocytogenes, and S. Typhimurium, separate test tubes containing 2 mL of TSB-YE were inoculated with a single, isolated bacterial colony, placed in a 37° C. incubator, and allowed to enrich with shaking. At various time periods a 500 μL sample of growth medium was collected from the tubes for each bacterium and analyzed using the paper device. Additionally, total plate counts, using tryptic soy agar with yeast extract (TSA-YE), were employed to obtain primary reference data for viable bacteria counts and for method validation. By analyzing each sample over several hours at set time intervals, the shortest enrichment time necessary for the determination of a pure culture was estimated for each assay. Since the composition of one transferred bacterial colony may vary from one to thousands of viable cells, the shortest enrichment time can only be approximated and could fluctuate depending on the number of cells present initially.

Limit of Detection.

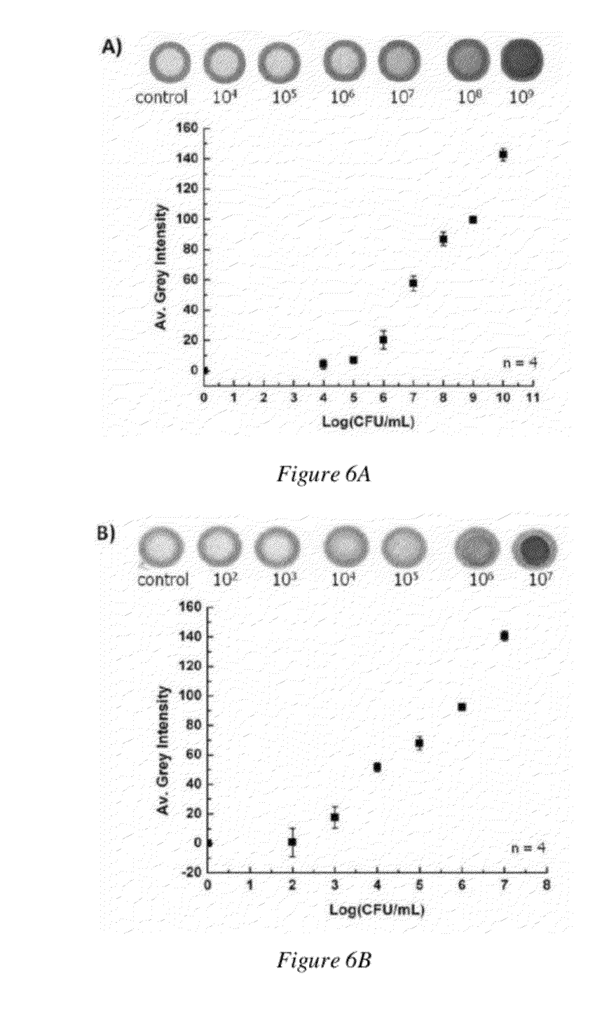

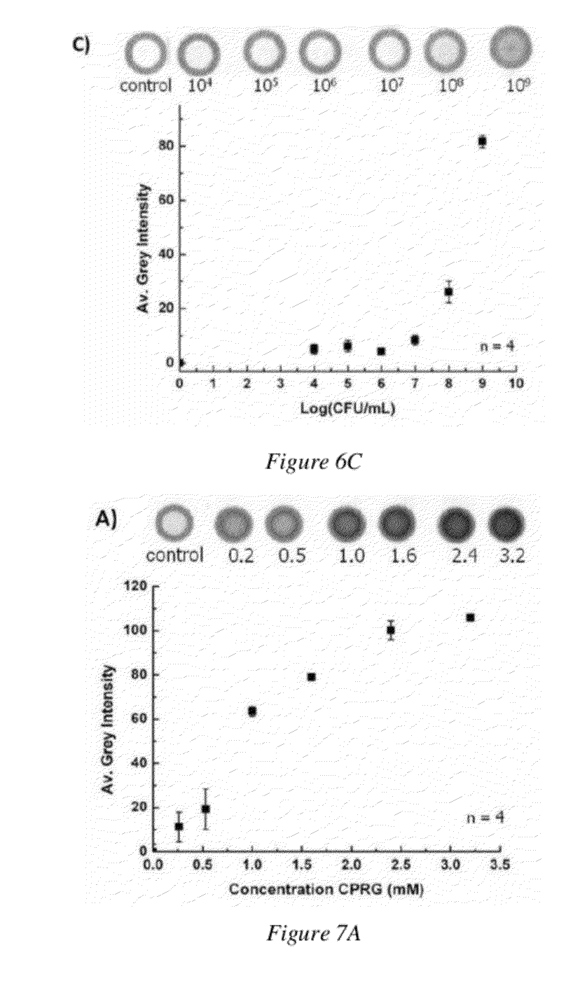

The limit of detection was determined for each assay. Isolated colonies were enriched for overnight to ensure a high concentration of cells, and serial dilutions were made in lambda buffer. A sample of each dilution was tested on the paper devices and plated for validation of bacterial cell concentration. The results of this study, including the grey intensity analysis, are shown in FIGS. 6A-6C. The limit of detection (LOD) for esterase occurs at 10⁴ CFU/mL concentration of S. Typhimurium, while the LODs for β-galactosidase and PIPLC occur at 10⁶ and 10⁸ CFU/mL, respectively. These studies provided a baseline for determining the concentration of bacteria necessary in the enrichment media to allow detection of bacteria from food samples. However, because the bacteria were enriched overnight, an accumulation of the target enzymes can be expected, and the measured enzyme activity from these samples does not directly correlate with the concentration of cells present. Differences in LODs are most likely due to differences in the expressed enzyme levels as well as the molar absorbtivities of the dyes used in these experiments.

Food Sample Analysis.

To demonstrate proof-of-concept, samples of bologna were inoculated with live bacteria and analyzed using the paper-based device. A 10 cm² area was marked with a permanent marker on each bologna sample. The samples were spot-inoculated with 10³ CFU/cm², 10² CFU/cm², and 10¹ CFU/cm² concentrations of live E. coli O157:H7, L. monoctyogenes, and S. Typhimurium. The initial concentrations of the bacteria were confirmed via plating onto TSA-YE following serial 10-fold dilutions in lambda buffer. The food samples were then placed in a sterile biosafety cabinet and allowed to dry for 3 hours. After the samples had dried, each 10 cm² area was swabbed thoroughly using the sampling swab from a Phast Swab device. [Willford, J. G. and Goodridge, L. D. Food Protection Trends 2008, 28, 468-472] The swab was placed directly into the Phast Swab reservoir containing 2 mL of TSB-YE. The tubes were placed in a 37° C. incubator and allowed to enrich with shaking. At various enrichment times an aliquot of each sample was tested on the μPAD for the presence of E. coli, L. monocytogenes, and S. Typhimurium and also plated using selective agar for method validation. Since these samples may contain a mixture of microorganisms, the use of selective and/or differential agars is necessary for satisfactory differentiation. Selective plating of E. coli O157:H7 was performed using MacConkey-sorbitol agar with CT supplement, PALCAM agar with supplement was used to selectively plate L. monocytogenes, and XLT-4 agar with Tergitol-4 supplement was used for plating S. Typhimurium. Both the enzymatic assay and plating results from spiked samples were compared with results from negative controls (bologna slices not inoculated with the target bacteria). All experiments involving live bacteria were carried out in a BSL-3 level biosafety cabinet using aseptic techniques to prevent infection. The results of the food sample analysis are presented in Example 7, below.

### Example 3

### Assay Development

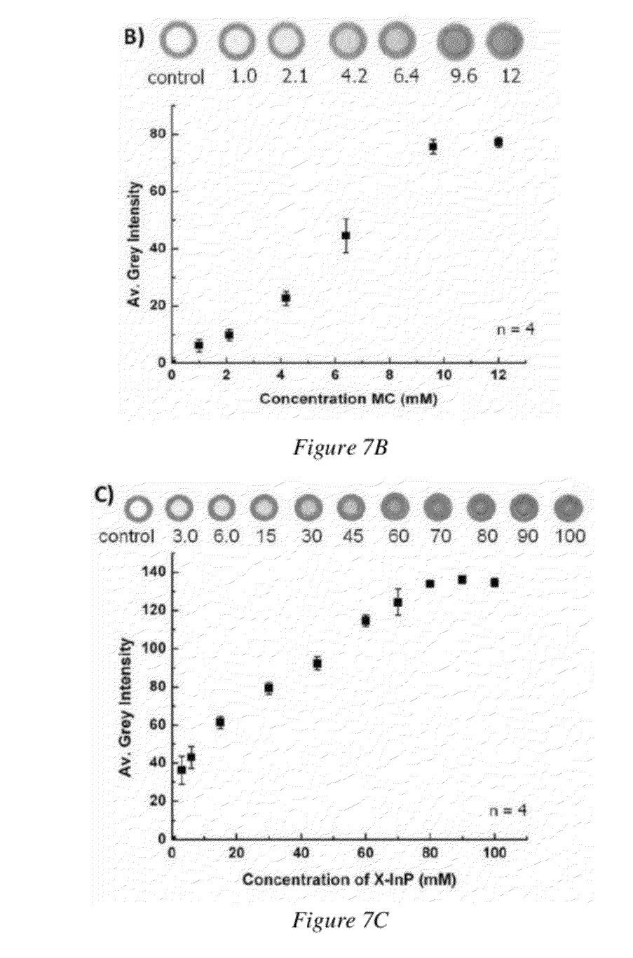

The optimal substrate concentration was established for each assay using only the enzyme (i.e. no live bacteria were used for this portion of the studies). Various concentrations of substrate/indicator reagent were added to the well device while the amount of enzyme and total volume of each well were held constant. The array of well devices was scanned after the enzymatic reactions were complete and wells had dried to generate a digital image and the grey intensity of each spot was measured. A plot of average grey intensity versus substrate concentration was generated, and a point of saturation for each assay was identified (FIGS. 7A-7C). The concentration of substrate at this saturation point was considered the optimal concentration for the system.

### Example 4

### Limit of Detection

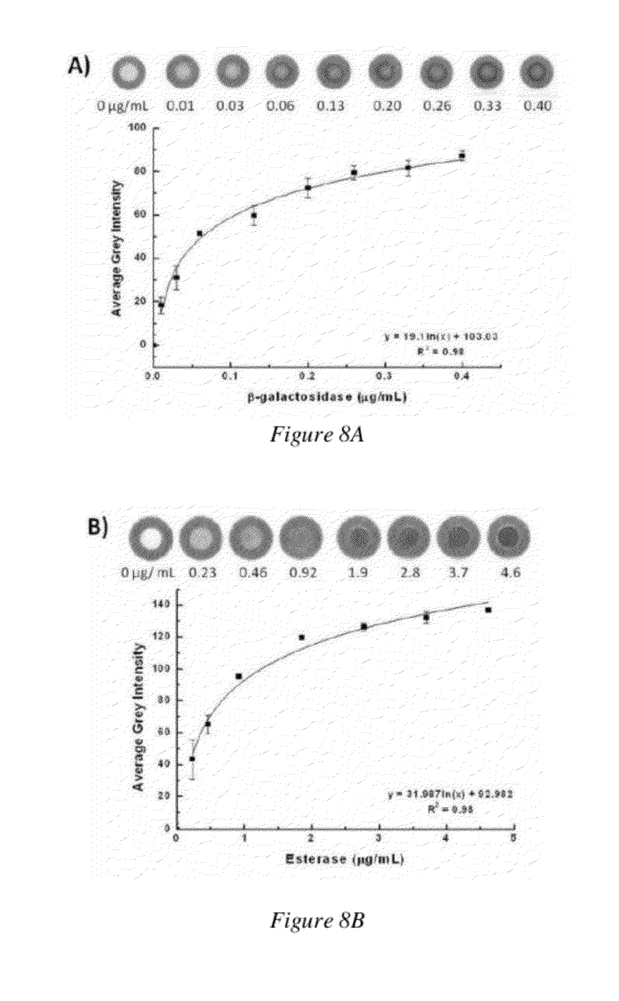

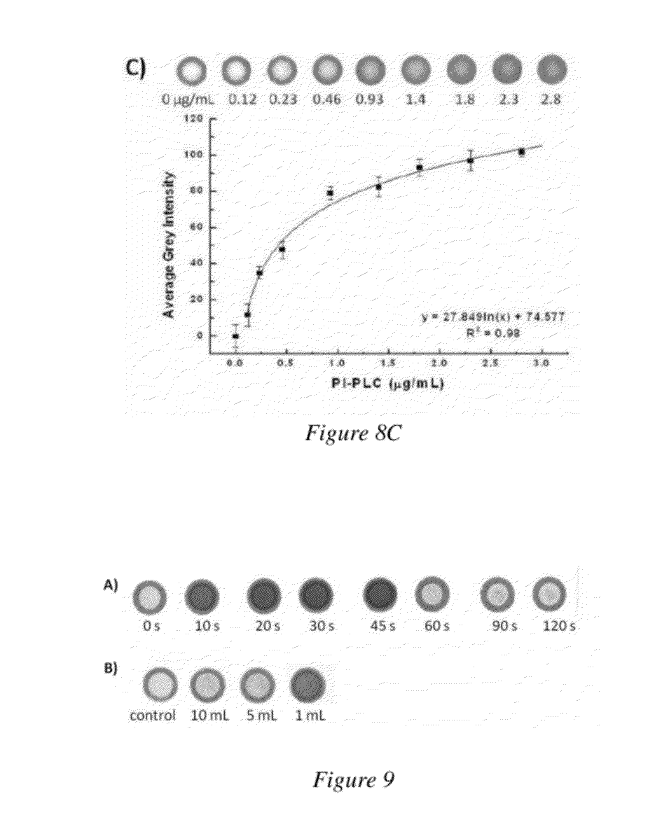

Using the optimal substrate concentrations, a limit of detection was determined for each enzyme (FIGS. 8A-8C). The substrate concentration was held constant while the concentration of enzyme decreased until no color formation was measured. The limit of detection, defined as the lowest detectable amount of enzyme that can be distinguished from the control, for β-galactosidase, esterase, and PI-PLC were 0.01±0.01 μg/mL, 0.23±0.08 μg/mL, and 0.12±0.08 μg/mL (n=4), respectively. A logarithmic trend is exhibited for each assay, which can be related to the measurement of reflectance from a limited surface area (7 mm diameter spot). Non-linear data correlations are common to colorimetric assays measured from digital images [Wang, S., et al., Lab Chip 2011, 11 (20), 3411-3418] and paper-based analytical devices, and are the result of surface saturation at high concentrations of product. [Li, X., et al., Anal. Bioanal. Chem. 2010, 396, 495-501; Steiner, M.-S., et al., Anal. Chem. 2010, 82, 8402-8405] Furthermore, in Michaelis-Menton enzyme kinetics, the reaction rate increases and asymptotically approaches the maximum velocity as the enzyme is saturated with substrate molecules. [Purich, D. L. Enzyme Kinetics: Catalysis & Control A Reference of Theory and Best-Practice Methods, Elsevier Inc., 2010]

### Example 5

### Analysis of Live Bacteria

Using pure cultures, each assay was optimized for analysis of live bacteria, with particular consideration paid to reducing the enrichment duration and investigating the need for cell lysis. In the determination of PI-PLC and esterase from L. monocytogenes and S. Typhimurium, respectively, the enzymes are either produced on the exterior of the cell or secreted by the cell into the growth media, allowing the enzymatic reactions to occur without the need to lyse cells. However, in the determination of β-galactosidase from E. coli O157:H7, the enzyme is generated inside the cell and is not secreted by the microorganism. Probe sonication was chosen as the lysis method because it provides fast, simple, and non-chemical cell rupture without denaturation of the target enzyme, and could easily be implemented in the field. Lysis of E. coli O157:H7 cells is relatively easy since the Gram-negative bacteria lack the rigid peptidoglycan layer in their cell wall. [Gannon, V. P., et al., Appl. Environ. Microbiol. 1992, 58, 3809-3815; Fykse, E. M., et al., J. Microbiol. Methods 2003, 55, 1-10] Samples of E. coli O157:H7 sonicated for 10 to 45 s produced the red-violet color change associated with the enzymatic hydrolysis of CPRG as shown in FIG. 9A. Sonication durations longer than 45 seconds did not produce a color change, most likely due to denaturation of the enzyme from extended sonication periods and/or the heat generated from the process. A sonication duration of 20 s was chosen for the remainder of the work because this time period allows for sufficient lysing of cells and agrees with other reports. [Fykse, E. M., et al., J. Microbiol. Methods 2003, 55, 1-10] Samples of L. monocytogenes and S. Typhimurium were also sonicated for 20 seconds and tested on the paper device to ensure sonication does not hinder the colorimetric detection of these species. At longer times, sonication inhibited the assay by denaturing the relevant enzymes.

In the determination and identification of live bacteria, current methods rely on a combination of cultural enrichment followed by biochemical and serological tests. [Zhu, P., et al., Biosens. Bioelectron. 2011, 30, 337-341; Bisha, B. and Brehm-Stecher, B. F. Appl. Environ. Microbiol. 2009, 75, 1450-1455] Enrichment media provides nutrients for bacteria, encouraging growth to the critical threshold concentration required for detection. Additionally, cultural enrichment can provide a level of selectivity when utilizing specific inhibiting and inducing supplements to allow for selective growth of a target species, while simultaneously suppressing the growth of competing microorganisms. It is proposed herein that that conducting a low-volume enrichment (e.g. <10 mL of media) aids in preconcentration of cells, and therefore, reduces the required incubation time, which, when combined with a streamlined detection system such as the PAD, can be used to rapidly and inexpensively detect pathogens in food and other environmental samples.

A sample of E. coli was diluted in 10, 5, and 1 mL TSB-YE and enriched for 5 hr. A 500 μL aliquot was collected from each sample, sonicated for 20 s, and tested on the paper device with CPRG. According to our hypothesis, a more intense color change was observed for the sample enriched in 1 mL growth media because this sample had a greater concentration of enzyme. The results of the colorimetric assay are shown in FIG. 9B.

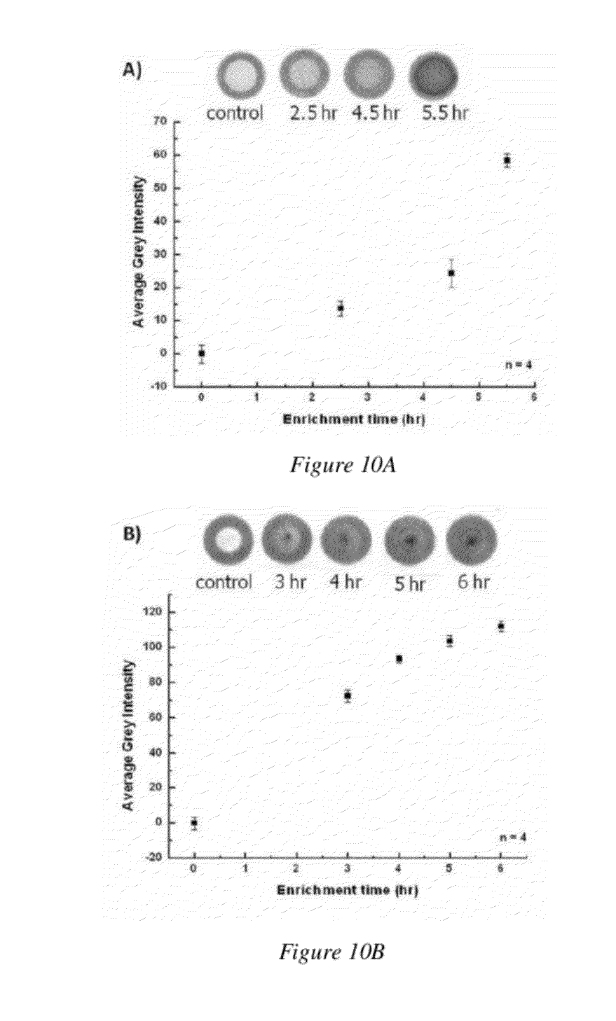

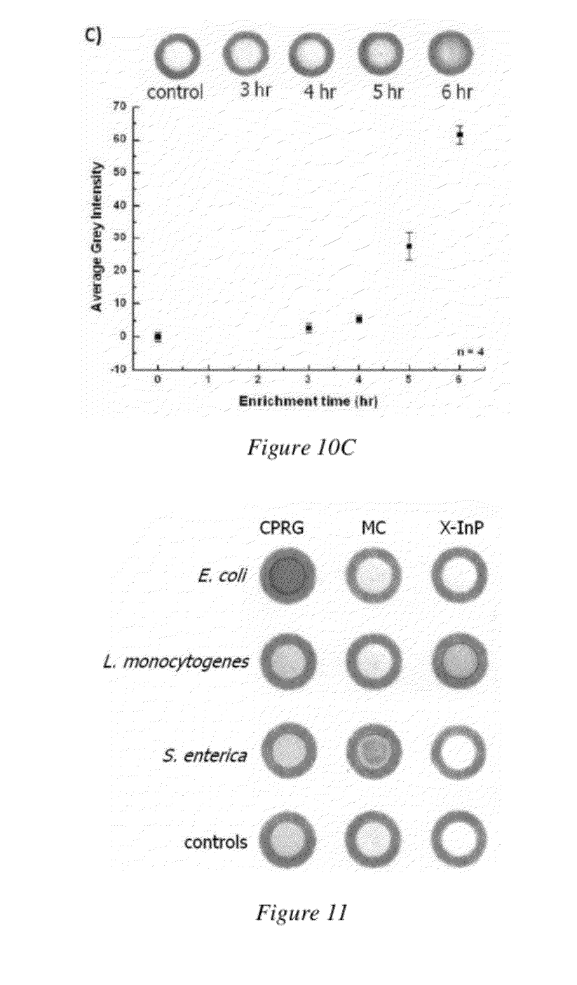

Using the low-volume enrichment strategy, inoculates of isolated bacterial cultures were tested on the paper devices at various enrichment time points to provide an estimate of the minimal enrichment time required for detection. The samples were also plated at each time point to confirm microbial numbers and validate the method. Pure culture of L. monocytogenes was detected after 5 hr of enrichment and the amount of PI-PLC enzyme detected was 0.18±0.08 μg/mL. E. coli O157:H7 was detected after 4.5 hr of enrichment, with 0.016±0.006 μg/mL β-galactosidase present. S. Typhimurium was detected after an enrichment period of only 3 hr, and the amount of esterase detected was 0.52±0.06 μg/mL. The results of the enrichment time study are shown in FIGS. 10A-10C (E. coli O157:H7 in FIG. 10A; L. monocytogenes in FIG. 10B; S. Typhimurium in FIG. 10C).

### Example 6

### Cross-Reactivity Testing

The assays utilized in this work involve enzymes that may be produced by multiple species of bacteria, and therefore, the cross-reactivity between the three assays was studied. The PI-PLC enzyme produced by L. monocytogenes is highly selective to this particular species (the only other species of Listeria to demonstrate PI-PLC activity is L. ivanovii); [Lauer, W. F., et al., J. AOAC Int. 2005, 88, 511-517; Notermans, S. H., et al., Appl. Environ. Microbiol. 1991, 57, 2666-2670; Vazquez-Boland, J. A., et al., Clin. Microbiol. Rev. 2001, 14, 584-640]. However, β-galactosidase is produced by many serotypes of E. coli in addition to O157:H7. [Manafi, M., et al., Microbiol. Mol. Biol. Rev. 1991, 55, 335-348] The selectivity of each assay was evaluated by performing a cross-reactivity study. In FIG. 11 an array of nine 7 mm wells on paper are presented. Each row was spotted with one of the three bacterial species in a concentration of approximately 10⁹ CFU/mL and each column was spotted with one of the three colorimetric substrates so that cross-reactivity among the different enzyme-substrate pairs could be analyzed. In all three cases, enzyme activity and color change were only observed when the correct enzyme-substrate pair was present, and none of the three bacterial species exhibited a false positive result.

### Example 7

### Detection of Pathogens from Inoculated Food Samples

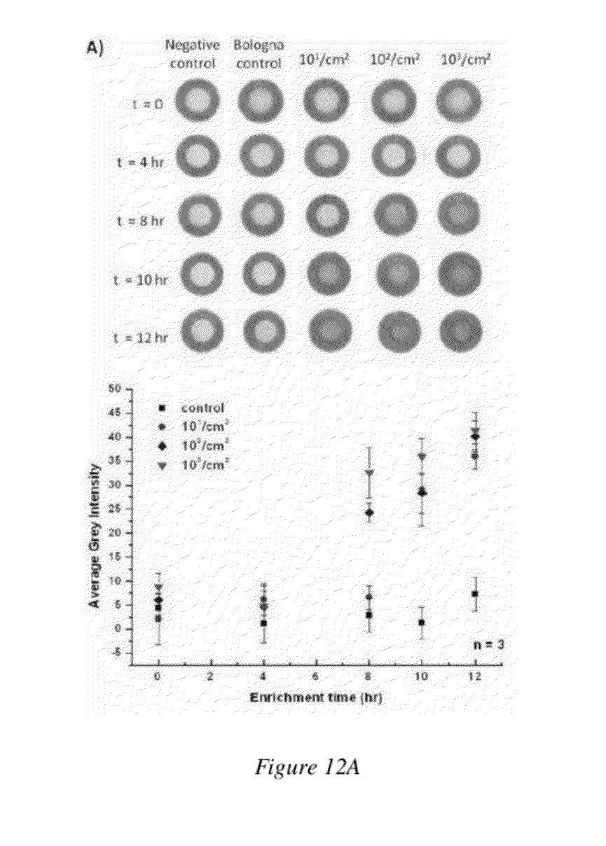

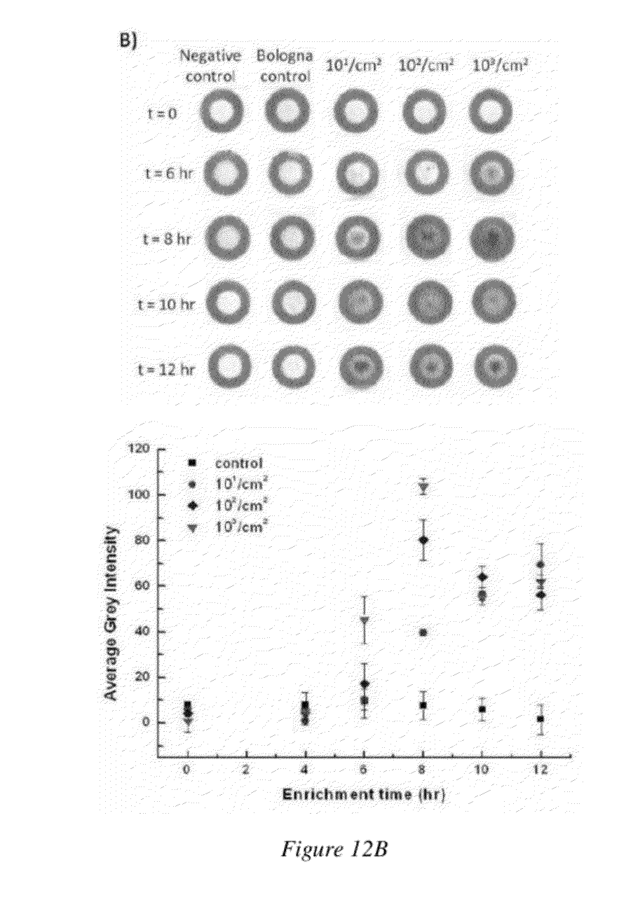

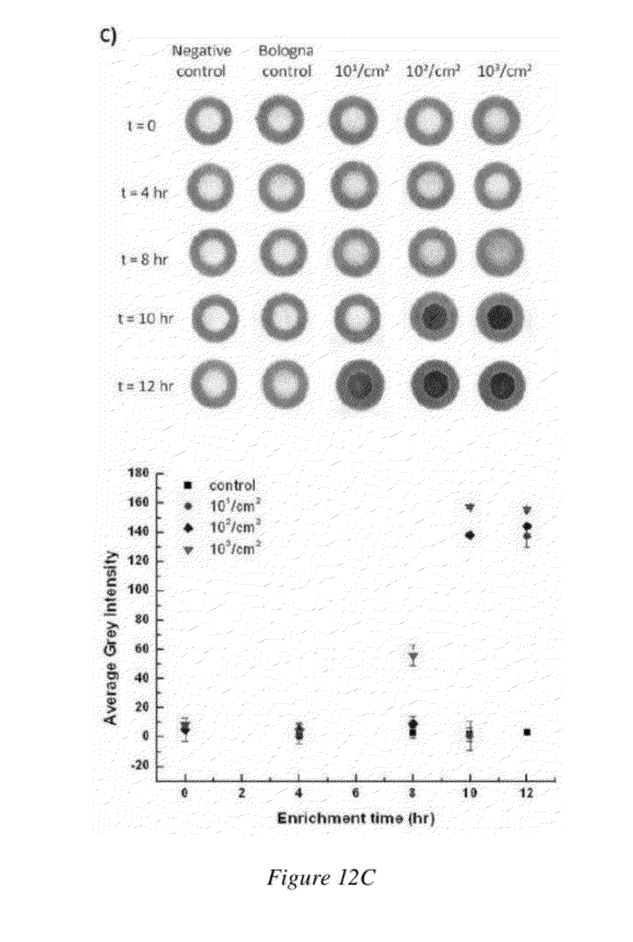

Individual ready-to-eat (RTE) meat samples were inoculated with 10³ CFU/cm², 10² CFU/cm², and 10¹ CFU/cm² E. coli O157:H7, L. monocytogenes, and S. Typhimurium to demonstrate proof-of-concept for real samples and the ability of the devices to detect low concentrations of pathogenic bacteria in real samples. In other words, each sample was inoculated with a single bacterial species at a single dilution. The surface of the bologna samples was swabbed to collect bacteria after a 3 hr drying period. The swab technique is less conventional than using a stomacher for sample preparation as it is strictly a surface sampling method; however, swabbing is fast, convenient, and easy to perform. [Willford, J. G. and Goodridge, L. D. Food Protection Trends 2008, 28, 468-472; Saumya, B. Molecular and Cellular Probes 2003, 17, 99-105] Swabs were placed directly in TSB-YE enrichment media, and aliquots of the media were tested at 0, 4, 8, 10, and 12 hr of enrichment. The 10¹ CFU/cm² concentration of the target bacterial species was detected within 8, 10, and 12 hr of enrichment for S. Typhimurium, E. coli O157:H7, and L. monocytogenes, respectively. The colorimetric results and corresponding grey intensity values for the three inoculated samples as well as controls are shown in FIGS. 12A-12C. Standard plating on selective agars was also performed to confirm the initial concentration of cells spiked onto RTE samples and to monitor the growth of target species throughout the enrichment process. Results from plating matched the results from the paper devices but required 48 hours to complete.

### Example 8

### Detection of Pathogens from Inoculated Water Samples

Surface water from the Cache La Poudre River in Northern Colorado was spiked with various concentrations of E. coli O157:H7 and S. Typhimurium. Seventy-eight samples were prepared and a blind study was conducted on the samples using the PAD devices. The testing utilized a single-step media enrichment.

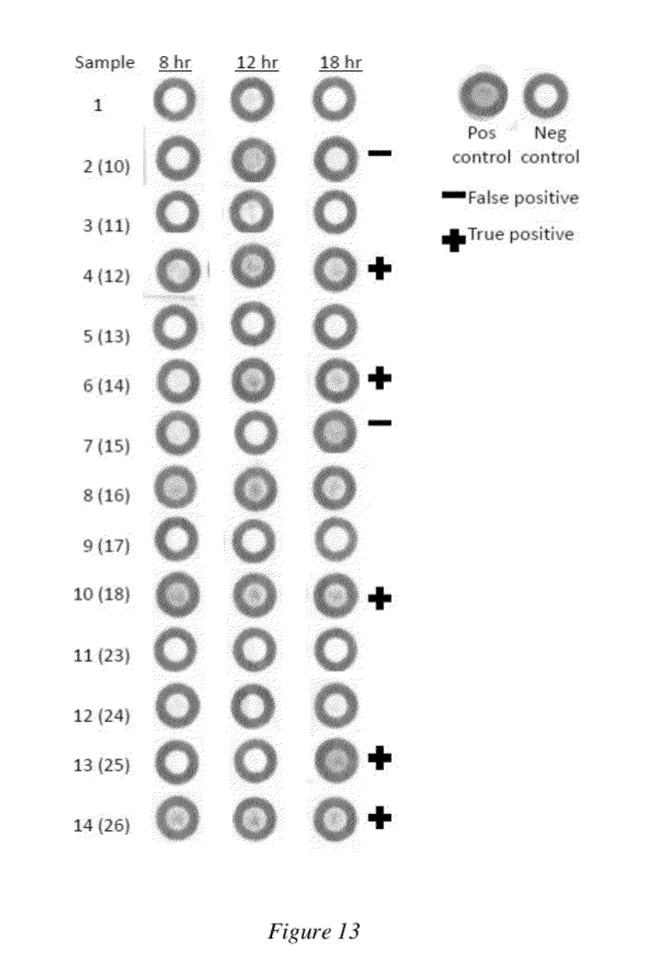

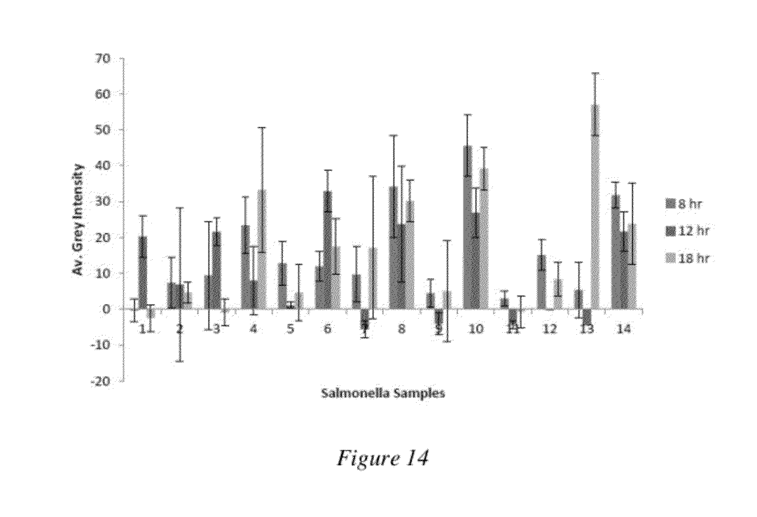

FIG. 13 shows an image of the resulting microspot analysis for S. Typhimurium of samples taken with the analysis performed following 8, 12 or 18 hours of enrichment. Qualitative analysis suggests samples 2, 4, 6, 7, 8, 10, 12, 13 & 14 tested positively. Further testing showed that all of these samples contain low levels of Salmonella except #2 (10) and #7 (15) (i.e. samples #2 (10) and #7 (15) were “false positives”). FIG. 14 is a graph showing ImageJ measurements on the samples presented in the microspot analysis shown in FIG. 13. Based on ImageJ measurements, samples 4, 6, 8, 10, 12, 13 & 14 tested positively.

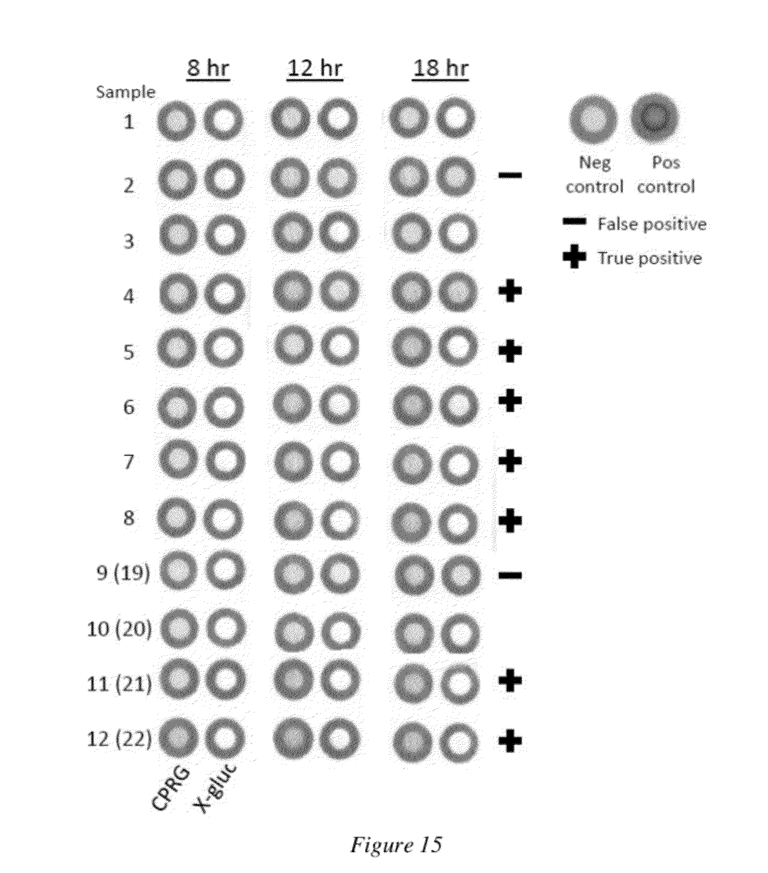

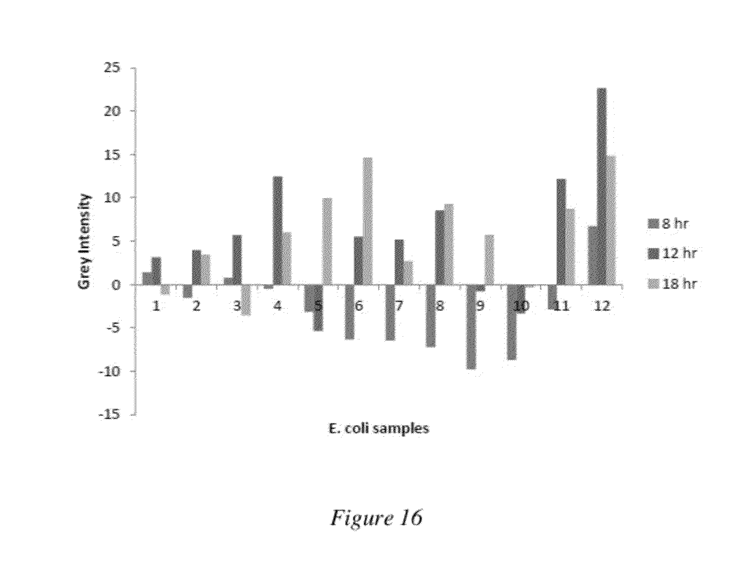

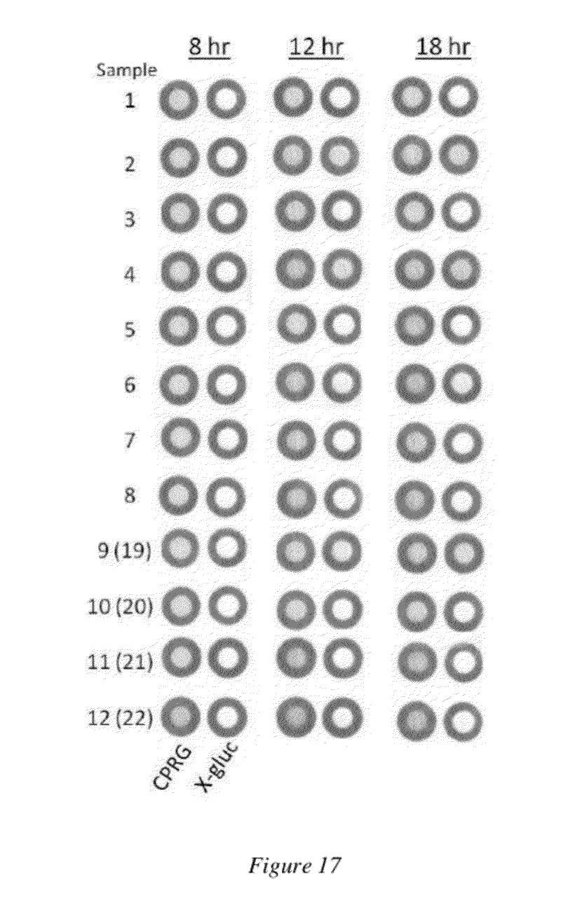

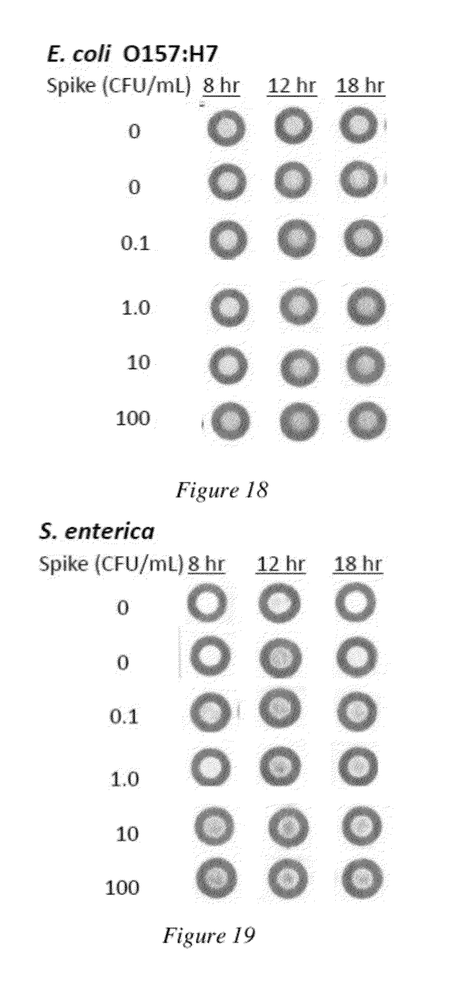

FIG. 15 shows an image of the resulting microspot analysis for E. coli of samples taken with the analysis performed following 8, 12 or 18 hours of enrichment. Qualitative analysis suggests samples 2, 4, 5, 6, 7, 8, 9, 11, and 12 tested positively (for CPRG). Further testing showed that all of these samples contain low levels of E. coli except samples 2 & 9 (19), which were false positives. FIG. 16 is a graph showing ImageJ measurements on the samples presented in the microspot analysis shown in FIG. 15. Based on ImageJ measurements, samples 2, 4, 5, 6, 7, 8, 9, 11, and 12 tested positively. However, samples 2 & 9 are false positives.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A kit for the detection of pathogens comprising:
an analytical device comprising:
a porous membrane having a first and second side;
a substantially continuous boundary deposited within the porous membrane extending from the first side to the second side defining the peripheral sides of a well or repository, the substantially continuous boundary comprised of a hydrophobic solid; and
a barrier adjacent to the first side of the membrane, wherein the barrier defines the bottom of the well and the second side of the membrane within the region defined by the substantially continuous boundary defines the top of the well;
one or more indicator reagents impregnated within the substantially continuous boundary of the membrane, wherein the indicator reagent produces a detectable change upon contact with a product of a pathogen of interest;
growth media, the growth media adapted to enrich a sample prior to assaying on the analytical device
and
instructions for use of the kit employing the analytical device and the one or more indicator reagents for the detection of a pathogen of interest.

2. The kit according to claim 1 wherein the indicator reagent is selected from the group consisting of 5-bromo-4-chloro-myo-inositol phosphate (X—InP), chlorophenyl red β-galactopyranoside (CPRG), 5-Bromo-4-chloro-3-indolyl-B-D-glucuronide (X-gluc) and 5-bromo-6-chloro-inositol caprylate (magenta caprylate).

3. The kit according to claim 1 wherein the membrane comprises a plurality of substantially continuous boundaries, thereby providing a plurality of wells on said membrane and wherein a plurality of indicator reagents are impregnated within the wells, each well of the plurality of wells impregnated with only one of the plurality indicator reagents, thereby allowing a plurality of bacterial species to be detected on a single membrane

4. The kit according to claim 1 wherein the porous membrane is selected from the group consisting of paper, nitrocellulose, polycarbonate, methylethyl cellulose, polyvinylidene fluoride (PVDF), polystyrene, and glass.

5. The kit according to claim 1 wherein the hydrophobic solid is selected from the group consisting of wax, photoresist, and solid ink.

6. The kit according to claim 1 wherein the instructions direct incubation of the sample in growth media for a time period selected from the group consisting of about 12 hours or less, about 10 hours or less, about 9 hours or less, about 8 hours or less, about 7 hours or less, about 6 hours or less, about 5 hours or less, about 4 hours or less, and about 3 hours or less.

7. A kit for the detection of pathogens comprising:
an analytical device comprising:
a porous membrane having a first and second side;
a substantially continuous boundary deposited within the porous membrane extending from the first side to the second side defining the peripheral sides of a well or repository, the substantially continuous boundary comprised of a hydrophobic solid; and
a barrier adjacent to the first side of the membrane, wherein the barrier defines the bottom of the well and the second side of the membrane within the region defined by the substantially continuous boundary defines the top of the well;
an indicator reagent, wherein the indicator reagent produces a detectable change upon contact with a product of a pathogen of interest; and
instructions for use of the kit employing the analytical device and the one or more indicator reagents for the detection of a pathogen of interest.

8. The kit according to claim 7 wherein the indicator reagent is impregnated within the substantially continuous boundary of the membrane.

9. The kit according to claim 7 wherein the indicator reagent is selected from the group consisting of 5-bromo-4-chloro-myo-inositol phosphate (X—InP), chlorophenyl red β-galactopyranoside (CPRG), 5-Bromo-4-chloro-3-indolyl-B-D-glucuronide (X-gluc) and 5-bromo-6-chloro-inositol caprylate (magenta caprylate).

10. The kit according to claim 7 wherein the indicator reagent reacts with an enzyme selected from the group consisting of β-galactosidase, esterase, glucoronidase, glucuronidase, and PI-PLC.

11. The kit according to claim 7 wherein the membrane comprises a plurality of substantially continuous boundaries, thereby providing a plurality of wells on said membrane.

12. The kit according to claim 11 wherein a plurality of indicator reagents are impregnated within a plurality of substantially continuous boundaries of the membrane, each well of the plurality of wells impregnated with only one of the plurality indicator reagents, thereby allowing a plurality of bacterial species to be detected on a single membrane.

13. The kit according to claim 7 wherein the porous membrane is selected from the group consisting of paper, nitrocellulose, polycarbonate, methylethyl cellulose, polyvinylidene fluoride (PVDF), polystyrene, and glass.

14. The kit according to claim 7 wherein the hydrophobic solid is selected from the group consisting of wax, photoresist, and solid ink.

15. The kit according to claim 7 further comprising growth media, the growth media adapted to enrich a sample prior to assaying on the analytical device.

16. The kit according to claim 7 wherein the instructions direct incubation of the sample in growth media for a time period selected from the group consisting of about 12 hours or less, about 10 hours or less, about 9 hours or less, about 8 hours or less, about 7 hours or less, about 6 hours or less, about 5 hours or less, about 4 hours or less, and about 3 hours or less.

17. A kit for the detection of L. monocytogenes comprising:
an analytical device comprising:
a porous membrane having a first and second side;
a substantially continuous boundary deposited within the porous membrane extending from the first side to the second side defining the peripheral sides of a well or repository, the substantially continuous boundary comprised of a hydrophobic solid; and
a barrier adjacent to the first side of the membrane, wherein the barrier defines the bottom of the well and the second side of the membrane within the region defined by the substantially continuous boundary defines the top of the well;
an indicator reagent impregnated within the substantially continuous boundary of the membrane, wherein the indicator reagent produces a detectable change upon contact with the enzyme PI-PLC of L. monocytogenes;
and
instructions for use of the kit employing the analytical device and the one or more indicator reagents for the detection of a pathogen of interest.

18. The kit according to claim 17 wherein the indicator reagent is X—InP.

19. The kit according to claim 17 further comprising growth media, the growth media adapted to enrich a sample prior to assaying on the analytical device.

20. The kit according to claim 19 wherein the instructions direct incubation of the sample in growth media for a time period selected from the group consisting of about 12 hours or less, about 10 hours or less, about 9 hours or less, about 8 hours or less, about 7 hours or less, about 6 hours or less, about 5 hours or less, about 4 hours or less, and about 3 hours or less.
